SEQUENCE LISTING (1 sequence)

SEQ ID NO: 1 - protein, 221 aa, synthetic construct
  REGION 1..221: note = Ancestral LPMO
VMKKMLMKSKKISIAVALLMAGLLTLLFASSASAHGYVSSPASRAYLCSLGKNTNCGSVQYEPQSVEAPKGFPAAGPADGQIASGGKFSQLDEQTANRWTKTPIKAGANTFTWHFTAPHATTKWHYYITKQGWNPNKPLTRADLELIPFCEIDDNGATPSTNVTHQVNVPSDRSGYHVILAVWDIADTGNAFYQVIDVNVGGGGATTAPANPTATGDKVTY

(19) United States
(12) Patent Application Publication
(10) Pub. No.: US 2024/0392259 A1
(43) Pub. Date: Nov. 28, 2024

(54) NOVEL LYTIC POLYSACCHARIDE MONOOXYGENASE AND USES THEREOF

(71) Applicant: ASOCIACIÓN CENTRO DE INVESTIGACIÓN COOPERATIVA EN NANOCIENCIAS "CIC NANOGUNE", San Sebastian, Guipuzcoa (ES)

(72) Inventors: Raúl PÉREZ JIMÉNEZ, San Sebastian, Guipuzcoa (ES); Borja ALONSO LERMA, San Sebastian, Guipuzcoa (ES); Leire BARANDIARAN LARREA, San Sebastian, Guipuzcoa (ES)

(21) Appl. No.: 18/691,224

(22) PCT Filed: Sep. 12, 2022
(86) PCT No.: PCT/EP2022/075229

§ 371 Date: Mar. 12, 2024

(30) Foreign Application Priority Data
Sep. 13, 2021 (EP) 21382819.7

(51) Int. Cl. C12N 9/02; C12P 19/26

(52) U.S. Cl. CPC C12N 9/0083; C12P 19/26; C12Y 114/99

(57) ABSTRACT

The invention relates to a polypeptide having lytic polysaccharide monooxygenase (LPMO) activity, well as to a nucleic acid encoding said polypeptide, and a vector and host cell comprising said nucleic acid. The invention also relates to a method for degrading a polysaccharide comprising β(1-4) glyosidic bonds and for producing crystalline nanochitin, as well as to the obtained crystalline nanochitin. The invention also relates to a composite material and a method for obtaining this composite material, and to the uses of the crystalline nanochitin and of the composite material.

The project leading to this application has received funding from the European Union's Horizon 2020 research and innovation program under grant agreement No 964764.”

### FIELD OF THE INVENTION

The present invention relates the field of enzymes, particularly, lytic polysaccharide monooxygenases, and their use for degrading chitin.

### BACKGROUND

In the biotechnology industry, enzymes are the basis of complex substrate transformations into valuable chemicals for a myriad of applications, ranging from bioenergy to food industry. Prominent examples are the enzymatic transformation of biomass into smaller molecules such as sugars, lignin and chitosan. Conversely, material science involves established chemical, physical and engineering procedures for transforming and designing novel materials, often by assembling small molecules used as building blocks. Interestingly, numerous enzymes have the potential for upgrading and even creating novel high-tech functional materials. However, this potential remains largely unexplored. Enzymes are generally used for specific transformations under given conditions. Additionally, enzyme design and search techniques have not concentrated on optimizing the interaction of enzymes with materials for transforming them into a distinctive material with properties unattainable by conventional non-selective chemical and physical treatments. Therefore, looking for enzymes with the ability to upgrade rather than degrade materials stands up like a challenge for enzyme design techniques.

Cellulose and chitin are well suited for enzyme transformation into high performance materials. They represent the first and second most abundant biopolymers on Earth, respectively. Cellulose is present in plants, while chitin is found in crustaceans, insects and fungi. Both have been suggested as the source of valuable materials for numerous applications. A vast number of enzymes have been described to act on biomass including cellulases, laccases, chitinases, transglutaminases, transaminases, expansins, among others. These enzymes promote fiber disruption and chemical functionalization in a very selective manner. Recently, a new class of oxidases was described, Lytic Polysaccharide Monooxygenase (LPMO), that catalyze the conversion of crystalline polysaccharides such as chitin and cellulose by oxidative cleavage of their β-(1→4) glycosidic bond, resulting in the selective oxidation of C1 and C4 carbons. LPMOs expanded the toolbox of biomass-acting enzymes being one of the few enzymes that can actually modify biomass physically and chemically, i.e., by fiber disruption and chemical functionalization.

Biomass is per se a recalcitrant material which often requires aggressive chemical and mechanical treatments, thus limiting the efficiency and usability of enzymes. In the past decades, the improvement of biomass-acting enzymes has been object of intense research, but mainly focusing on improving their efficiency towards degrading biomass.

Therefore, there is a need for new LPMO enzymes.

### SUMMARY OF THE INVENTION

The inventors have designed a new form of ancient LPMO using Ancestral Sequence Reconstruction. Using a vast collection of bacterial LPMOs, the inventors reconstructed an LPMO sequence from the Last Firmicutes/Actinobacteria Common Ancestor (LFACA), about 3 billion years old. This ancestral LFACA-LPMO has advantageous properties compared to modern LPMOs, such as high temperature and pH stabilities (FIGS. 2b and 2c). Also, LFACA-LPMO was able to degrade the complex structure of chitin (FIG. 1) into smaller fibers and nanofibers, much more efficiently than its modern counterpart from Bacillus thurigiensis (BtLPMO) (FIG. 2d). However, the most important feature of the new LPMO is that the enzymatic crystalline nanochitin produced (EnCNCh) showed physicochemical modifications that enhanced its ability to serve as a stable functionalized biomaterial for cell growth in 2D and 3D cultures. These results demonstrate that EnCNCh acts as versatile bioink that can also incorporate graphene derivatives. Given the growing importance of chitin in the biotechnology industry, EnCNCh emerges as a promising material for biomedical applications.

Accordingly, in a first aspect, the invention relates to a polypeptide having lytic polysaccharide monooxygenase (LPMO) activity comprising the sequence of SEQ ID NO: 1 or a functionally equivalent variant thereof, wherein the functionally equivalent variant thereof has at least 65% sequence identity to the sequence of SEQ ID NO: 1 throughout the whole length of SEQ ID NO: 1.

In a second aspect, the invention relates to a nucleic acid encoding the polypeptide of the first aspect, or to a vector comprising said nucleic acid, or to a host cell comprising said nucleic acid or said vector.

In a third aspect, the invention relates to a method for degrading a polysaccharide comprising a β(1-4) glyosidic bond comprising contacting a sample containing said polysaccharide with a copper-bound polypeptide having lytic polysaccharide monooxygenase (LPMO) activity under suitable conditions for the oxidative cleavage of said β(1-4) glycosidic bond, wherein the polypeptide having LPMO activity is the polypeptide of the first aspect.

In a fourth aspect, the invention relates to a method for producing crystalline nanochitin comprising contacting a sample containing chitin with a copper-bound polypeptide having lytic polysaccharide monooxygenase (LPMO) activity under suitable conditions for the oxidative cleavage of chitin, wherein the polypeptide having LPMO activity is the polypeptide of the first aspect.

In another aspect, the invention relates to the use of the crystalline nanochitin of the fifth aspect as a bioink for 3D bioprinting or as a substrate for cell growth. In another aspect, the invention relates to a composite material comprising:
(a) crystalline nanochitin, wherein said crystalline nanochitin is obtained by a method comprising contacting a sample containing chitin with a copper-bound polypeptide having lytic polysaccharide monooxygenase activity (LPMO) under suitable conditions for the oxidative cleavage of chitin; and
(b) a carbon material selected from the group consisting of carbon nanotubes, carbon nanofibers, graphene, graphene oxide and a mixture thereof.

In another aspect, the invention relates to a method for the preparation of the composite material of the seventh aspect, comprising the steps of:
(i) preparing a suspension comprising crystalline nanochitin and a carbon material selected from the group consisting of carbon nanotubes, carbon nanofibers, graphene, graphene oxide and a mixture thereof;
(ii) mixing the suspension; and
(iii) optionally reducing said carbon material in the mixture of the previous step.
   In another aspect, the invention relates to a composite material obtainable by the method according to the eighth aspect.

In another aspect, the invention relates to the use of the composite material of the ninth aspect as a bioink for 3D bioprinting or as a substrate for cell growth.

### DESCRIPTION OF THE DRAWINGS

FIG. 1. α-Chitin enzymatic hydrolysis. The hierarchical structure of chitin allows its degradation into nanoscale particles. To obtain chitin nanoparticles enzymatic hydrolysis was performed in water at 50° C., 72 hours and 10 mg enzyme per gram of substrate. Ascorbic acid 2 mM was used as electron donor. It is shown how LPMO affinity to chitin fibbers catalyses the degradation of this fiber into smaller fibers. Controlling hydrolysis time, nanochitin with different sizes can be obtained. Chitin nanocrystals (CNCh) are small crystalline particles with lengths between 100 and 500 nm and diameters between 3 and 40 nm.

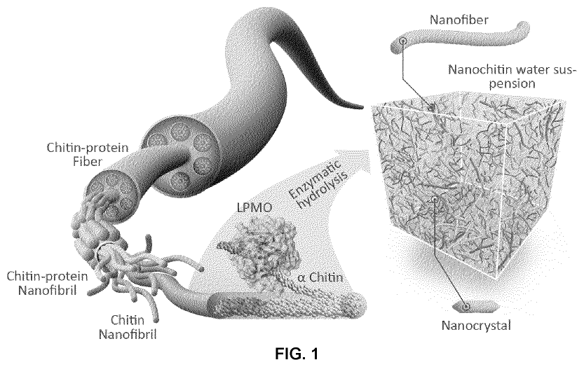

FIG. 2. Ancestral LPMO reconstruction and characterization. (a) Uncorrelated lognormal relaxed-clock chronogram of LPMO with geological time inferred with Bayesian inference. A total of 51 LPMO homologous sequences were used. UniProt identification codes can be found in Table 1. Divergence times were estimated using Bayesian inference and information from the Time Tree of Life. Geological scale and times are indicated in the upper bar. The selected internal node for prior analysis is shown with a circle LFACA (Last Firmicutes Actinobacteria Common Ancestor). Geological times are shown in the upper bar. (b) LFACA LPMO and Bt LPMO temperature assays from 30° C. to 90° C. (c) LPMOs pH activity assay from pH 3 to pH 10. LFACA LPMO and Bt LPMO show low stability at alkaline pHs but their activity increases at pH 5, almost reaching its optimum pH, which is at pH 6. LFACA LPMO keeps increasing its activity until pH 8, where it reached the maximum activity value. At pH 9 the LFACA LPMO maintains over 80% of its activity whereas extant LPMO maintains 50% of its optimal activity. (d) Nanochitin production at different times after LFACA LPMO and Bt LPMO activity.

FIG. 3. Nanochitin morphology (a) AFM images of nanoparticles produced by LPMO hydrolysis at different times. The images have 3×3 microns dimensions and show the nanoparticles obtained from the α-chitin hydrolysis with ancestral LPMO (Anc ENCNCh), query Bacillus thurigiensis LPMO (Q EnCNCh) and HCl (Ac CNCh) at times of 24, 48, 72, and 96 hours. (b) Size distribution of CNCh particles produced with Anc LPMO, Q CNCh and Ac CNCh hydrolysis using α-chitin as substrate. Size distribution was calculated with the length of 100 particles from each condition.

FIG. 4. Nanochitin physicochemical characterization. (a) FTIR spectra of CNCh produced by oxidative cleavage of α-chitin with LFACA LPMO (Anc EnCNCh), Bt LPMO (Q EnCNCh) and Hydrochloric acid (AcCNCh). Spectra from 4000 to 750 cm⁻¹. (b) Details of the significant incipient peaks of CNCh at 1710 cm⁻¹, C═O vibration region for carboxylic acid from amide I CO at 1621 cm⁻¹, and C—O stretching and bending shape of the peak changing detail at 1070 and 1008 cm⁻¹. (c) CP/MAS ¹³C NMR spectra of chitin, LFACA EnCNCh produced with LFACA LPMO enzymatic oxidative cleavage, Bt EnCNCh produces with extant Bacillus thurigiensis LPMO oxidative cleavage and AcCNCh produced by hydrochloric acid hydrolysis. (d) Thermogravimetric analysis curves of chitin, LFACA EnCNCh (red) produced with LFACA LPMO enzymatic oxidative cleavage, Bt EnCNCh (blue) produces with Bt LPMO enzymatic oxidative cleavage and AcCNCh (yellow) produced by hydrochloric acid hydrolysis. Inset of weight loss curves and derivative from TGA curves. (e) XRD diffractogram for crystalline CNCh (CNCh) obtained by LFACA LPMO oxidative cleavage (LFACA EnCNCh), Bt LPMO (Bt EnCNCh) and with HCl (AcCNCh) and α-chitin.

FIG. 5. Cell culture on nanochitin. (a) Image of EnCNCh cylindric scaffold 3D printing, where HEK293 cells are seeded. (b) Confocal microscopy image at 10× and (c) 100× of HEK293T stained nuclei with DAPI and cell membrane with Orange Plasma. Cells proliferated embedded on EnCNCh matrix after 3 days of culture. (d) Picture of EnCNCh film where HEK293T cells are grown (e) confocal microscopy image at 10× and (f) 100× of HEK293T stained with DAPI and Orange Plasma seeded and grown on the film. (g) Scheme of EnCNCh film where S. aureus bacteria do not grow after 24 hours of incubation. (h) Confocal images of EnCNCh film after incubating S. aureus.

FIG. 6. EnCNCh/rGO films fabrication. (a) Detail of CNCh films from left to right: AcCNCh film, EnCNCh film, EnCNCh film+1% wt rGO, 5% wt rGO and 10 wt rGO. EnCNCh is transparent and had good integrity unlike AcCNCh that was very weak and not as translucent (b) Conductivity of EnCNCh for different reduced graphene contents: 0.3, 1, 2, 5, 10 and 15 wt % rGO. Calculations were made with volumetric % of reduced graphene oxide. Inset a log-log plot is shown for the determination of electrical percolation threshold of the material (P_{c}). (c) Image of 3D EnCNCh+5% GO 3D printing of a stable honeycomb like scaffold. (d) Picture of a EnCNCh+5% GO 3D printed bottom shape scaffold where HEK293 cells are grown. (e) Confocal microscopy image at ×10 of HEK293T-stained nuclei with DAPI and Orange Plasma seeded and grown on the hybrid material.

FIG. 7. Posterior probability of all the inferred residues of the LFACA LPMO (Ancestral LPMO). The residue with the highest posterior probability is assigned at each position and average posterior probability value for the inferred LFACA LPMO is above 0.9.

FIG. 8. (a) SDS-PAGE electrophoresis of purified LFACA LPMO and Bt LPMO. A band at around 20 kDa is observed in both cases, indicating the correct size of the proteins. A higher production of LFACA LPMO using the exact same culture volume and expression conditions was also observed. (b). LPMO production. LFACA is produced at higher quantity than Bt LPMO, around 25% more protein is recovered after purification from 100 mL culture.

FIG. 9. Size distribution of CNCh particles produced with (a) LFACA LPMO in red and (b) Bt LPMO in blue using α-chitin as substrate. The longer the hydrolysis the darker the line gets in the graphs for both enzymes. The distribution was calculated with the length of 100 particles from each condition.

FIG. 10. FTIR spectra of EnCNCh produced by LFACA LPMO at 24, 48, 72 and 96 hours hydrolysis of α-chitin. (a) Spectra from 4000 to 750 cm⁻¹, (b) from 2000 to 750 cm⁻¹ and (c) Details of the significant incipient peaks of EnCNCh at 1710 cm⁻¹ and C—O stretching and bending peak shape changing detail at 1070 and 1008 cm⁻¹.

FIG. 11. FTIR spectra of EnCNCh produced by Bt LPMO at 24, 48, 72 and 96 hours hydrolysis of α-chitin. (a) Spectra from 4000 to 750 cm⁻¹, (b) amplification from 2000 to 750 cm⁻¹ and (c) details of the significant incipient peaks of CNCh at 1710 cm⁻¹ and C—O stretching and bending shape changing detail at 1070 and 1008 cm⁻¹.

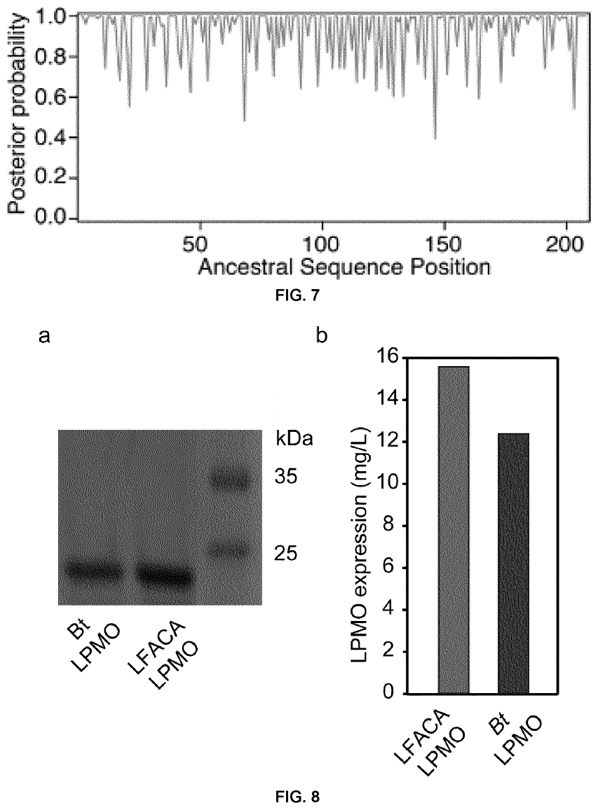

FIG. 12. CNCh films immersion into water. After immersion films are test the integrity of the film in water by orbital agitation. AcCNCh film did not maintain shape and disintegrated shortly after sinking into water whereas EnCNCh maintained their shape.

FIG. 13. EnCNCh+n % GO films pictures. From left to right rGO wt % increases.

FIG. 14. SEM images of films prepared with EnCNCh with different graphene oxide concentrations: 0.3, 2, 5, 10 and 15 wt %. The surface of films seems rougher when rGO content increases.

FIG. 15. FTIR spectra of EnCNCh and EnCNCh+n % GO films. Spectra from 4000 to 750 cm⁻¹

FIG. 16. (a) Representation of 2D EnCNCh+5% rGO paper. (b) Graphic representation of HEK293 cells seeded on top of 2D EnCNCh+5% rGO paper. (c) Confocal microscopy image at 10× and 100× of HEK293T stained nuclei with DAPI and cell membrane with Orange Plasma. Cells proliferated embedded on EnCNCh+5% rGO 2D paper after 3 days of culture.

### DETAILED DESCRIPTION OF THE INVENTION

### Polypeptide of the Invention

In a first aspect, the invention relates to a polypeptide having lytic polysaccharide monooxygenase (LPMO) activity comprising the sequence of SEQ ID NO: 1 or a functionally equivalent variant thereof, wherein the functionally equivalent variant thereof has at least 65% sequence identity to the sequence of SEQ ID NO: 1 throughout the whole length of SEQ ID NO: 1.

The term “polypeptide”, as used herein refers to a chain of amino acids of any length wherein the different amino acids are linked to one another by means of peptide bonds or disulphide bridges.

The term “lytic polysaccharide monooxygenase” or “LPMO”, as used herein, refers to a class of copper-dependent enzymes that cleave polysaccharides through an oxidative mechanism. LPMO are classified as Auxiliary Activity (AA) enzymes by the Carbohydrate-Active enZymes database (CAZy; http://www.cazy.org). The proposed mechanism of action consists of the cleavage of the polysaccharide by the insertion of oxygen at C1 and/or C4, with the subsequent formation of a lactone, which is spontaneously hydrolyzed to aldonic acid or a ketoaldose, respectively. LPMO have been classified in the CAZy auxiliary activities AA9, AA10, AA11, and AA13 (Levasseur A. et al., Biotechnol Biofuels. 2013; 6:41), and in the preliminary EC classification 1.14.99.B6. LPMO catalyze the oxidative depolymerization of diverse polymeric carbohydrates such as cellulose (AA9, AA10), hemicellulose (AA9, AA14]), chitin (AA10, AA11), and starch (AA13).

In a particular embodiment, the polypeptide of the invention comprises or consists of or essentially consist of the amino acid sequence of SEQ ID NO: 1.

In a particular embodiment, the polypeptide of the invention comprises or consists of or essentially consists of a functionally equivalent variant of the amino acid sequence of SEQ ID NO: that substantially maintains its catalytic activity.

The term “functionally equivalent variant” as used herein is understood to mean all those proteins derived from a sequence by modification, insertion and/or deletion or one or more amino acids, whenever the function is substantially maintained. In a particular embodiment, the functionally equivalent has LPMO activity. In another particular embodiment, the functionally equivalent variant substantially maintains the catalytic activity of the polypeptide of SEQ ID NO: 1.

In a particular embodiment, the variant results from the substitution of one or more amino acids. Said substitution may be a conservative substitution, which in general indicates that one amino acid is substituted with another amino acid having similar properties. Conservative substitution tables providing functionally similar amino acids are well known in the art. The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (1), Leucine (L), Methionine (M), Valine (V); and
6) Phenylalanine (F), Tyrosine (Y), Tryptophan (W).

In particular, the functionally equivalent variant of the LPMO of SEQ ID NO: 1, substantially maintains the catalytic activity of the LPMO from which it derived.

The term “catalytic activity” or “enzyme activity”, as used herein, refers to the ability of an enzyme to accelerate or catalyse chemical reactions. The catalytic activity is a measure of the quantity of active enzyme present and is thus dependent on reaction conditions, including temperature and/or pH, which should be specified. The commonly used unit is enzyme unit (U)=1 μmol min⁻¹. Another common unit is the specific activity of an enzyme, which is the activity of an enzyme per milligram of total protein (expressed in μmol min⁻¹ mg⁻¹) and measures enzyme purity in the mixture.

The catalytic activity is characterised by means of the following kinetic parameters: Vₘₐₓ, which is the maximum speed of an enzymatic reaction; the Michaelis-Menten constant (Kₘ), which is the substrate concentration required for an enzyme to reach one-half its maximum reaction rate; and k_{cat}, or turnover number, which is the number of substrate molecules handled by one active site per second. These kinetic parameters depend on solution conditions, such as temperature and pH, and on substrate concentration. The efficiency of an enzyme can be expressed in terms of k_{cat}/Kₘ, or specificity constant. Because the specificity constant reflects both affinity and catalytic ability, it is useful for comparing different enzymes against each other, or the same enzyme with different substrates.

The term “LPMO activity”, as used herein, refers to the catalytic activity of the LPMO.

The term “catalytic activity of the LPMO of SEQ ID NO: 1”, as used herein, refers to the ability of this sequence to catalyse the oxidative cleavage of (1-4)-β-D-glucosidic links at C1 or C4 on polysaccharides.

The LPMO catalytic activity may be measured by means of a number of techniques assays that are conventional to the skilled person, including the assay used in the examples, which is a spectrophotometric assay that measures LPMO peroxidase activity and was described by Breslmayr, E. et al., Biotechnology for biofuels 2018, 11 (1), 1-13. In this assay the substrate 2,6-dimethoxyphenol (2,6-DMP) and the cosubstrate H₂O₂ are converted to the 2,6-DMP radical and water. Then tow 2,6-DMP phenoxy radicals dimerize and form hydrocoerulignone, which is again oxidized by LPMO to the chromogenic product coerulignone.

Other examples of assays that can be sued for measuring LPMO activity include the following:
Methods based on HPLC quantification of the reaction products from different substrates, such as cellulose (Forsberg Z. et al., Protein Sci. 2011; 20:1479-83), hemicellulose (Agger J W et al., Proc Natl Acad Sci USA. 2014; 111:6287-92) and chitin (Loose JSM et al., FEBS Lett. 2014; 588:3435-40).
Methods based on nuclear magnetic resonance quantification of the reaction products from different substrates (Westereng B. et al., J Chromatogr A. 2013; 1271:144-52).
Methods based on the analysis with carbohydrate gel electrophoresis (PACE) of the activity of LPMO on PASC (phosphoric acid swollen cellulose) (Frandsen KEH et al., Nat Chem Biol. 2016; 12:298-303).
Methods based on the FRET quenching detection of the cleavage of a fluorescence-labeled cellotetraose (Frandsen KEH et al., Nat Chem Biol. 2016; 12:298-303).
A microarray method to detect LPMO activity on hemicellulose (Agger J W et al., Proc Natl Acad Sci USA. 2014; 111:6287-92).
A method based on micro-plate-based detection of insoluble C1-oxidized products of LPMO by fluorescence labelling with 7-amino-1,3-naphthalene-disulfonic acid (ANDA) (Kračun SK et al., Biotechnol Biofuels 2015; 8:70).

According to the present invention, the catalytic activity of the LPMO of sequence with SEQ ID NO: 1 is substantially maintained if the functionally equivalent has at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% of the catalytic activity of the LPMO of SEQ ID NO: 1 or if the functionally equivalent variant has at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, or at least 65%, at least 70%, at least 75%, at least 80%, at least 85%, at least 90%, at least 95%, at least 100%, at least 150%, at least 200%, at least 300%, at least 400%, at least 500%, at least 1000%, or more of the catalytic activity of LPMO of SEQ ID NO: 1.

As with other enzymes, the LPMO catalytic activity depends on a number of reaction parameters, including temperature and pH. Thus, in one embodiment, the functionally equivalent variant of the LPMO comprising, consisting essentially of or consisting of the sequence SEQ ID NO: 1 substantially maintains its catalytic activity at a temperature of at least 0° C., at least 5° C., at least 10° C., at least 15° C., at least 20° C., at least 25° C., at least 30° C., at least 35° C., at least 37° C., at least 40° C., at least 45° C., at least 50° C., at least 55° C., at least 60° C., at least 65° C., at least 70° C., at least 75° C., at least 80° C., at least 85° C., at least 90° C., at least 95° C., at least 100° C., or higher. Likewise, in another embodiment the functionally equivalent variant of the LPMO comprising, consisting essentially of or consisting of the sequence SEQ ID NO: 1 maintains its catalytic activity at pH 0, or at least pH 0.1, or at least pH 0.5, or at least pH 1.0, or at least pH 1.5, or at least pH 2.0, or at least pH 2.5, or at least pH 3.0, or at least pH 3.5, or at least pH 4.0, or at least pH 4.5, or at least pH 5.0, or at least pH 5.5, or at least pH 6.0, or at least pH 6.5, or at least pH 7.0, or at least pH 7.5, or at least pH 8.0, or at least pH 8.5, or at least pH 9.0, or at least pH 9.5, or at least pH 10.0, or at least pH 10.5, or at least pH 11.0, or at least pH 11.5, or at least pH 12.0, or at least pH 12.5, or at least pH 13.0, or at least pH 13.5, or pH 14. All possible combinations of temperatures and pH are also contemplated by the invention.

In particular embodiment, the functionally equivalent variant of the LPMO of SEQ ID NO: 1 that substantially maintains its catalytic activity has at least 65%, at least 70%, at least 75%, at least 80%, at least 85%, at least 90%, at least 93%, at least 95%, at least 97%, at least 98% or at least 99% sequence identity with SEQ ID NO: 1. In a more embodiment, the functionally equivalent variant of the LPMO of SEQ ID NO: 1 that substantially maintains its catalytic activity has at least 65% sequence identity with SEQ ID NO: 1.

The degree of identity between the variants and the LPMO having the sequence of SEQ ID NO: 1 is determined by using algorithms and computer methods which are widely known by the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J Mol Biol, 215: 403-410 (1990)]. The sequence identity is determined throughout the whole length of the sequence of the LPMO of sequence SEQ ID NO: 1, or through the whole length of the variant or both.

In a particular embodiment, the functionally equivalent variant of the LPMO of sequence SEQ ID NO: 1 does not have the sequence of the LMPOs included in Table 1.

In a particular embodiment, the polypeptide of the invention further comprises a tag suitable for detection and/or purification located at the N-terminus or at the C-terminus.

The polypeptide can be purified from the medium or from the cell lysate by means of affinity to commercial molecules showing a high affinity for said tags.

The term “tag”, as used herein, refers to any amino acid sequence for which specific binding molecules are available, thus allowing the detection/purification of any polypeptide carrying said tag. The tag is generally placed at the amino- or the carboxyl-terminus of the polypeptide. The presence of such tag allows the adapter molecule to be detected using an antibody against the tag polypeptide. Also, the provision of the tag enables the adapter polypeptide to be readily purified by affinity purification using an anti-tag antibody or another type of affinity reagent that binds to the epitope tag.

Suitable detection/purification tags include hexa-histidines (metal chelate moiety), affinity for hexa-hat GST (glutathione S-transferase) glutathione, calmodulin-binding peptide (CBP), streptomycin tag, cellulose-binding domain, maltose-binding protein, S-peptide tag, chitin-binding tag, immunoreactive epitopes, epitope tags, E2tag, HA epitope tag, Myc epitope, FLAG epitope, AU1 and AU5 epitopes, Glu-Glu epitope, KT3 epitope, IRS epitope, Btag epitope, protein kinase-C epitope, VSV epitope or any other tag provided that the tag does not affect the stability of the protein. In a preferred embodiment, the tag is hexa-histidine. Additional tag polypeptides and their respective antibodies are well known in the art. Illustrative, non-limitative examples are poly-histidine-glycine (poly-his-gly) tags; the flu HA tag polypeptide and its antibody 12CA5; the c-myc tag and the 8F9, 3C7, 6E10, G4, B7 and 9E10 antibodies; the Herpes Simplex virus glycoprotein D (gD) tag and its antibody. Other tag polypeptides include tubulin epitope peptide; and the T7 gene 10 protein peptide tag. In a particular embodiment, the polypeptide of the invention comprises a poly-histidine tag.

In a particular embodiment, the polypeptide of the invention is copper-bound. The term “copper-bound”, as used herein, refers to the presence of copper ions conjugated with the polypeptide of the invention. Without wanting to be bound to any particular theory, the polypeptide of the invention has LPMO activity and therefore, like all the LPMO enzymes, it requires the presence of a copper ion in its active site.

### Nucleic Acid, Vector and Host Cell of the Invention

In another aspect, the invention relates to a nucleic acid encoding the polypeptide of the invention.

The term “nucleic acid”, as used herein, relates to a deoxyribonucleotide or ribonucleotide polymer in either single or double stranded form and, unless otherwise limited, encompasses natural nucleotides and analogues of natural nucleotides that hybridize to nucleic acids in a manner similar to naturally occurring nucleotides. The term “nucleotide” includes, but is not limited to, a monomer that includes a base (such as a pyrimidine, purine or synthetic analogs thereof) linked to a sugar (such as ribose, deoxyribose or synthetic analogs thereof), or a base linked to an amino acid, as in a peptide nucleic acid (PNA). A nucleotide is one monomer in an oligonucleotide or in a polynucleotide. A “nucleotide sequence” or “nucleic acid sequence” refers to the sequence of bases in an oligonucleotide or in a polynucleotide.

Different hosts often have preferences for a particular codon to be used for encoding a particular amino acid residue. Such codon preferences are well known and a DNA sequence encoding a desired fusion protein sequence can be altered, using in vitro mutagenesis for example, so that host-preferred codons are utilized for a particular host in which the fusion protein is to be expressed.

In a particular embodiment, the nucleic acid further comprises a sequence encoding a signal peptide fused in frame at the 5′ terminus. The term “signal peptide”, as used herein, also known as signal, localization signal, localization sequence, transit peptide, leader sequence or leader peptide refers to a short peptide present at the N-terminus of the majority of newly synthesized proteins that are destined towards the secretory pathway. “In frame” or “operatively linked”, as used herein, means that the nucleic acid of the invention and the signal peptide are expressed in the correct reading frame under control of the expression control or regulating sequences.

In another aspect, the invention relates to a vector comprising the nucleic acid of the invention.

The term “vector”, as used herein, refers to a nucleic acid sequence comprising the necessary sequences so that after transcribing and translating said sequences in a cell the first or second polypeptide of the invention is generated. Said sequence is operably linked to additional segments that provide for its autonomous replication in a host cell of interest. Preferably, the vector is an expression vector, which is defined as a vector, which in addition to the regions of the autonomous replication in a host cell, contains regions operably linked to the nucleic acid of the invention and which are capable of enhancing the expression of the products of the nucleic acid according to the invention. The vectors of the invention can be obtained by means of techniques widely known in the art.

Any vector containing a host-compatible promoter, origin of replication and termination sequences is suitable.

A person skilled in the art will understand that there is no limitation as regards the type of vector which can be used because said vector can be a cloning vector suitable for propagation and for obtaining the polynucleotides or suitable gene constructs or expression vectors in different heterologous organisms suitable for purifying the conjugates. Thus, suitable vectors according to the present invention include prokaryotic expression vectors (e.g. pUC18, pUC19, Bluescript and their derivatives), mp18, mp19, pBR322, pMB9, ColEI, pCRI, RP4, phages and shuttle vectors (e.g. pSA3 and pAT28), yeast expression vectors (e.g. vectors of the type of 2 micron vectors), integration vectors, YEP vectors, centromeric vectors and the like, insect cell expression vectors (e.g. the pAC series and pVL series vectors), plant expression vectors, such as vectors of expression in plants (e.g. plBI, pEarleyGate, pAVA, pCAMBIA, pGSA, pGWB, pMDC, pMY, pORE series vectors), and eukaryotic expression vectors based on viral vectors (e.g. adenoviruses, viruses associated to adenoviruses as well as retroviruses and lentiviruses), as well as non-viral vectors (e.g. pSilencer 4.1-CMV (Ambion®, Life Technologies Corp., Carlsbad, CA, US), pcDNA3, pcDNA3.1/hyg pHCMV/Zeo, pCR3.1, pEFI/His, pIND/GS, pRc/HCMV2, pSV40/Zeo2, pTRACER-HCMV, pUB6/V5-His, pVAXI, pZeoSV2, pCl, pSVL and pKSV-10, pBPV-1, pML2d and pTDTI).

Vectors may further contain one or more selectable marker sequences suitable for use in the identification of cells which have or have not been transformed or transfected with the vector. Markers include, for example, genes encoding proteins which increase or decrease either resistance or sensitivity to antibiotics or other compounds (e.g. hyg encoding hygromycin resistance), genes which encode enzymes whose activities are detectable by standard assays known in the art (e.g. β-galactosidase or luciferase), and genes which visibly affect the phenotype of transformed or transfected cells, hosts, colonies or plaques such as various fluorescent proteins (e.g. green fluorescent protein, GFP). Alternatively, the vectors of the present invention may carry a non-antibiotic selection marker, including, for instance, genes encoding a catabolic enzyme which enables the growth in medium containing a substrate of said catabolic enzyme as a carbon source. An example of such a catabolic enzyme includes, but is not restricted to, lacYZ encoding lactose uptake and beta-galactosidase. Other selection markers that provide a metabolic advantage in defined media include, but are not restricted to, galTK for galactose utilization, sacPA for sucrose utilization, trePAR for trehalose utilization and xylAB for xylose utilization. Alternatively, the selection can involve the use of antisense mRNA to inhibit a toxic allele, for instance the sacB allele.

In another aspect, the invention relates to a host cell comprising the nucleic acid of the invention or the vector of the invention.

The term “host cell”, as used herein, refers to a cell into which a nucleic acid of the invention, such as a polynucleotide or a vector according to the invention, has been introduced and is capable of expressing the polynucleotides of the invention. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Host cells suitable for the expression of the nucleic acid or vector of the invention include, without being limited thereto, cells from bacteria, fungi, plants, insects and mammals. Bacterial cells include, without being limited thereto, cells from Gram-positive bacteria, such as species from the genera Bacillus, Streptomyces and Staphylococcus, and cells from Gram-negative bacteria, such as cells from the genera Escherichia and Pseudomonas. Fungi cells preferably include cells from yeasts such as Saccharomyces, Pichia pastoris and Hansenula polymorpha. Insect cells include, without limitation, Drosophila cells and Sf9 cells. Plant cells include, amongst others, cells from cultivated plants, such as cereals, medicinal plants, ornamental plants or bulbs. Mammalian cells suitable for this invention include epithelial cell lines (porcine, etc.), osteosarcoma cell lines (human, etc.), neuroblastoma cell lines (human, etc.), epithelial carcinomas (human, etc.), glial cells (murine, etc.), hepatic cell lines (from monkeys, etc.), CHO (Chinese Hamster Ovary) cells, COS cells, BHK cells, HeLa, 911, AT1080, A549, 293 or PER.C6 cells, human NTERA-2 ECC cells, D3 cells from the mESC line, human embryonary stem cells, such as HS293 and BGV01, SHEF1, SHEF2 and HS181, NIH3T3, 293T, REH and MCF-7 cells, and hMSC cells.

In a preferred embodiment the host cell is a bacterium, more preferably E. coli.

### Method for Degrading a Polysaccharide Comprising a β(1-4) Glyosidic Bond

In another aspect, the invention relates to a method for degrading a polysaccharide comprising a β(1-4) glyosidic bond, hereinafter first method of the invention, said method comprising contacting a sample containing said polysaccharide with a copper-bound polypeptide having LPMO activity under suitable conditions for the oxidative cleavage of said β(1-4) glycosidic bond, wherein the polypeptide having LPMO activity is the polypeptide of the invention.

The term “polysaccharide” as used herein, refers to a chain of monosaccharide units linked through glycosidic bonds. Polysaccharides can be homopolysaccharides, if all the monosaccharides in the polysaccharide are the same, or heteropolysaccharides, formed by different types of monosaccharides.

The term “β(1-4) glyosidic bond”, as used herein, refers to the covalent boding of oxygen to the carbon 1 of a saccharide and the carbon 4 of other saccharide, having the linked carbons a different stereochemistry.

Illustrative non-limitative examples of polysaccharides comprising β(1-4) glyosidic bonds include cellulose, hemicellulose and chitin.

In a particular embodiment, the polysaccharide comprising a β(1-4) glyosidic bond is selected from the group consisting of cellulose, hemicellulose and chitin. In a more particular embodiment, the polysaccharide comprising a β(1-4) glyosidic bond is chitin.

The term “cellulose”, as used herein, refers to an organic compound with CAS number 9004-34-6, a polysaccharide consisting of a linear chain of several hundred to many thousands of β(1→4) linked D-glucose units. The term “cellulose” includes the six polymorphs I, II, III, IV_{I} and IV_{II}, and also cellulose Iα and cellulose Iβ. The cellulose can include both amorphous and crystalline cellulose.

The term “hemicellulose”, as used herein, refer to a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. Hemicelluloses include xylans, mannans, mixed linkage β-glucans and xyloglucans. Xylans usually consists of a backbone of β(1→4) linked xylose residues. Manans include galactomamans (β(1→4) linked D-mannopyranose residues in linear chains) and glucomannans (both β(1→4) linked D-mannopyranose and β(1→4) linked D-glucopyranose residues in the main chains). Mixed β-glucans contain blocks of β-(1→4) D-Glucopyranose separated by single β-(1→3) D-Glucopyranose. Xyloglucans have a backbone similar to cellulose with α-D-Xylopyranose residues at position 6.

The term “chitin”, as used herein, relates to a β(1-4) polymer of N-acetyl-D-glucosamine that is the major structural component of the exoskeleton of invertebrates, cuticles of insects and the cell walls of fungi. Chitin is a linear, highly crystalline homo polymer of β-1,4 N-acetyl glucosamine (GIcNAc), that consists of β-1,4-linked N-acetyl glucosamine residues that are arranged in antiparallel (α), parallel (β) or mixed (γ, two parallel strands alternate with a single anti-parallel strand) strands, with the (α) configuration being the most abundant. In most organisms, chitin is cross-linked to other structural components, such as proteins and glucans. Chitin is represented by the following formula:

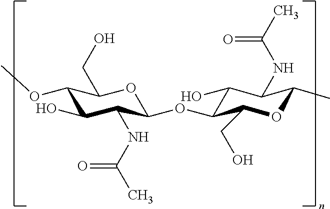

Each polymeric chitin chain is associated with neighboring chain by hydrogen bond, where amino group of one molecule makes bond with carbonyl group of the adjacent one. Naturally, chitin is found in three crystalline polymorphic forms having different orientations of the microfibrils: α-chitin, which has antiparallel chains, β-chitin, which has parallel chains and γ-chitin, which has a mixture of antiparallel and parallel chains, where, out of each three chitin chains, two are arranged in parallel fashion and one is arranged in antiparallel fashion. α-chitin is considered to be the most stable form of chitin.

In a particular embodiment, the chitin is selected from the group consisting of α-chitin, β-chitin and γ-chitin. In a more particular embodiment, the chitin is of α-chitin.

Natural chitin has a high degree of crystallinity but also contains amorphous domains.

The term “sample containing a polysaccharide comprising a β(1-4) glyosidic bond”, as used herein, refers to any type of sample comprising said polysaccharide, for example a sample containing cellulose, such as lignocellulose biomass, corn stover, Panicum virgatum (switchgrass), Miscanthus grass species, wood chips and the byproducts of lawn and tree maintenance; or a sample containing chitin. Chitin can be obtained from natural resources such as wastes from marine aqua animal shells and from the cell wall of fungus. In a particular embodiment, the sample comprises a pure polysaccharide comprising a β(1-4) glyosidic bond, such as pure cellulose or pure chitin. In a more particular embodiment, the sample is a water dissolution of α-chitin.

The first method of the invention comprises contacting the polypeptide of the invention with a polysaccharide comprising a β(1-4) glyosidic bond. The polypeptide of the invention has been previously defined. All the particular and prefer embodiments of the polypeptide of the invention apply to the method of the invention. In a particular embodiment, the polypeptide of the invention comprises or consists of the amino acid sequence of SEQ ID NO: 1.

The first method of the invention comprises contacting the polypeptide of the invention with a polysaccharide comprising a β(1-4) glyosidic bond under suitable conditions for the oxidative cleavage of said β(1-4) glyosidic bond, wherein the polypeptide of the invention is bound to copper.

The term “copper-bound” has been previously defined.

The term “copper ions”, as used herein, refers to Cu⁺¹ and Cu⁺². In a particular embodiment, the copper ions are Cu⁺².

The polypeptide of the invention, preferably the polypeptide comprising or consisting of SEQ ID NO: 1, is contacted with the copper ions before said polypeptide is contacted with the polysaccharide. The polypeptide of the invention can be contacted with a saturated solution of a salt of a copper ion, for example Cu_{(II)}SO₄. In a particular embodiment, the polypeptide of the invention, preferably the polypeptide comprising or consisting of SEQ ID NO: 1, is bound to copper by incubation with a saturated solution of a salt of a copper ion, such as Cu_{(II)}SO₄ for 30 minutes at room temperature, and at a ratio protein: salt of 1:3.

In a preferred embodiment, the contact between the sample comprising a β(1-4) glyosidic bond polysaccharide and the copper-bound polypeptide of the invention, preferably the polypeptide comprising or consisting of SEQ ID NO: 1, is done in the absence of free copper in the solution. Therefore, after the polypeptide of the invention, preferably the polypeptide comprising or consisting of SEQ ID NO: 1, is bound to copper by incubation with a saturated solution of a salt of a copper ion, the non-binded copper is removed from the solution by any suitable technique, for example, size exclusion chromatography.

The term “suitable conditions for the oxidative cleavage of said β(1-4) glycosidic bond” refers to any conditions under which a said β(1-4) glycosidic bond, preferably chitin β(1-4) glycosidic bond, can be cleaved by the polypeptide of the invention, preferably the polypeptide comprising or consisting of SEQ ID NO: 1. Such conditions are known by the skilled person. As a way of illustrative non-limitative example, the polypeptide of the invention, preferably the polypeptide comprising or consisting of SEQ ID NO: 1, is combined with a sample containing the polysaccharide, preferably a water solution of chitin, in the presence of cupper ions.

In a particular embodiment, the sample comprising the polysaccharide, preferably chitin, and the polypeptide of the invention, preferably the polypeptide comprising or consisting of SEQ ID NO: 1, are contacted under a pH between 5 and 10, particularly, about 5.5, about 6.0, about 6.5, about 6.8, about 7.0, about 7.2, about 7.4, about 7.6, about 7.8, about 8.0, about 8.5, about 9.0, about 9.5, about 9.8, more particularly between 6.5 and 7.5.

In a particular embodiment, the sample comprising the polysaccharide, preferably chitin, and the polypeptide of the invention, preferably the polypeptide comprising or consisting of SEQ ID NO: 1, are contacted under a temperature between 25° C. and 90° C., between 30° C. and 90° C., between 40° C. and 60° C., between 45° C. and 55° C., for example approximately 30° C., approximately 35° C., approximately 40° C., approximately 45° C., approximately 50° C., approximately 55° C., approximately 60° C., approximately 65° C., approximately 70° C., approximately 75° C., approximately 80° C., preferably approximately 50° C.

In a particular embodiment, the sample comprising the polysaccharide, preferably chitin, and the polypeptide of the invention, preferably the polypeptide comprising or consisting of SEQ ID NO: 1, are contacted for a period of time of at least 1 hour, 2 hours, 4 hours, 5 hours, 6 hours, 8 hours, 10 hours, 12 hours, 24 hours, 36 hours, 48 hours, 60 hours, 72 hours, 84 hours, 96 hours, 108 hours, 120 hours or more.

In a particular embodiment, the sample comprising the polysaccharide, preferably chitin, and the polypeptide of the invention, preferably the polypeptide comprising or consisting of SEQ ID NO: 1, are contacted under agitation.

In a particular embodiment, the first method of the invention further comprises contacting the sample comprising the polysaccharide, preferably chitin, and the polypeptide of the invention, preferably the polypeptide comprising or consisting of SEQ ID NO: 1, in the presence of a reducing agent.

The term “reducing agent”, as used herein, refers to a reagent which can donate electrons in an oxidation-reduction reaction. Suitable reducing agents that can be used in the first method of the invention include malonic acid, ascorbic acid, carbonic acid, citric acid, succinic acid, hydrogen peroxide or other suitable dicarboxylic acids, ketones or diketones, and mixtures thereof. In a particular embodiment, the reducing agent is ascorbic acid.

In a particular embodiment, when the polysaccharide is chitin, the first method can further comprise contacting the sample comprising chitin with a chitinase.

The term “chitinase” as used herein, refers to glycosyl hydrolases (E.C. 3.2.2.14) that can degrade chitin directly to low molecular weight chitooligomers. Chitinases have been divided into 2 main groups: Endochitinases (E.C 3.2.1.14) and exo-chitinases. The endochitinases randomly split chitin at internal sites, thereby forming the dimer di- cetylchitobiose and soluble low molecular mass multimers of GIcNAc such as chitotriose, and chitotetraose. The exo-chitinases have been further divided into 2 subcategories: Chitobiosidases (E.C. 3.2.1.29), which are involved in catalyzing the progressive release of di-acetylchitobiose starting at the non-reducing end of the chitin microfibril, and 1-4-β-glucosaminidases (E.C. 3.2.1.30), cleaving the oligomeric products of endochitinases and chitobiosidases, thereby generating monomers of GIcNAc. Illustrative non-limitative examples of chitinases that can be used in the first method of the invention include chitinases from bacteria, fungus, plants, insects and mammals. Illustrative non-limitative of chitinases that can be sued include chitinases from Streptomyces, Alteromonas, Escherichia and Aeromonas.

The sample comprising chitin can be contacted with the chitinase before, after or at the same time the sample is contacted with the polypeptide of the invention, preferably the polypeptide comprising or consisting of SEQ ID NO: 1.

In a particular embodiment, when the polysaccharide is cellulose, the first method of the invention can further comprise contacting the sample comprising cellulose with a cellulase.

The term “cellulase”, as used herein, refer to a group of enzymes that catalyze the hydrolysis of cellulose and include:
Endocellulases or endoglucanases (EC 3.2.1.4): enzyme that randomly cleaves (1-4)-β-D-glucosidic links in cellulose, lichenin and cereal β-D-glucans, thereby creating new chain ends. Endocellulases also hydrolyse 1,4-linkages in β-D-glucans also containing 1,3-linkages.
Exocellulases or exocellulases (EC 3.3.1.91): enzyme that catalyses the hydrolysis of β(1-4)-linkages in 1,4-beta-D-glucans and related oligosaccharides, removing two to four units from the ends of the exposed chains of polysaccharides releasing tetrasaccharides, disaccharides and some monosaccharides
Beta-glucosidases or cellobiases (EC 3.2.1.21): enzyme that catalyses the hydrolysis of β(1-4) bonds linking two or four glucose or glucose-substituted molecules (i.e., the disaccharide cellobiose or the tetrasaccharide cellotetraose) releasing glucose.

In a particular embodiment, the cellulase is selected from the group consisting of an endocellulase, an exocellulase, a beta-glucosidase and a combination thereof.

The sample comprising cellulose can be contacted with the glycosidase before, after or at the same time the sample is contacted with the polypeptide of the invention, preferably the polypeptidea comprising or consisting of SEQ ID NO: 1.

### Method for Producing Crystalline Nanochitin

In another aspect the invention refers to a method for producing crystalline nanochitin, hereinafter second method of the invention, comprising contacting a sample containing chitin with a copper-bound polypeptide having lytic polysaccharide monooxygenase (LPMO) activity under suitable conditions for the oxidative cleave of chitin, wherein the polypeptide having LPMO activity is the polypeptide of the invention.

In a particular embodiment, the polypeptide having LPMO activity is, the polypeptide comprising or consisting of SEQ ID NO: 1.

The terms “sample”, “chitin” and copper ions have been previously defined. The particular and preferred embodiments of the first method of the invention regarding these terms fully apply to the second method of the invention. In a particular embodiment the chitin is α-chitin. In a particular embodiment, the sample is a water solution of chitin, preferably α-chitin. In a particular embodiment, the copper ions are Cu²⁺.

The term “crystalline nanochitin” or “chitin nanocrystals” or “ChNC” or “chitin nanowhiskers” as used herein, refers to still rod-like nanoparticles consisting of chitin chain segments with very high crystallinity. ChNC have normally an average diameter between 2 nm to 20 nm and a length of 50 nm to 300 nm, as opposed to chitin nanofibers (ChNF), which are long thin fibers with a length of 1000 nm to 5000 and diameters ranging from 10 nm to 50 nm, and include both amorphous and crystalline domains. Another difference between CNC and CNF is the aspect ratio (length/diameter). It is generally accepted that ChNF have an aspect ratio over 100, while the aspect ratio of ChNC is generally lower.

The suitable conditions for the oxidative cleavage of chitin are the same previously defined for the oxidative cleavage of the β(1-4) glyosidic bond in the first method of the invention.

Without wanting to be bound to any theory, it is understood that LPMOs, and as such also the polypeptide of the invention, cleave the amorphous regions of chitin more easily than the crystalline domains. Therefore, depending on the conditions under which the sample comprising chitin and the enzyme are contacted, nanochitin particles of different length, diameter, aspect/ratio and crystallinity can be obtained in different proportions.

In a particular embodiment, the sample comprising chitin and the polypeptide of the invention, preferably the polypeptide comprising or consisting of SEQ ID NO: 1, are contacted under a pH between 5 and 10, particularly, about 5.5, about 6.0, about 6.5, about 6.8, about 7.0, about 7.2, about 7.4, about 7.6, about 7.8, about 8.0, about 8.5, about 9.0, about 9.5, about 9.8, more particularly between 6.5 and 7.5.

In a particular embodiment, the sample comprising chitin and the polypeptide of the invention, preferably the polypeptide comprising or consisting of SEQ ID NO: 1, are contacted under a temperature between 25° C. and 90° C., between 30° C. and 70° C., between 40° C. and 60° C., between 45° C. and 55° C., for example approximately 30° C., approximately 35° C., approximately 40° C., approximately 45° C., approximately 50° C., approximately 55° C., approximately 60° C., approximately 65° C., approximately 70° C., approximately 75° C., approximately 80° C., preferably approximately 50° C.

In a particular embodiment, the sample comprising chitin and the polypeptide of the invention, preferably the polypeptide comprising or consisting of SEQ ID NO: 1, are contacted for a period of time between 24 and 96 hours, for example, 24 hours, 26 hours, 28 hours, 30 hours, 32 hours, 34 hours, 36 hours, 38 hours, 40 hours, 42 hours, 44 hours, 46 hours, 48 hours, 60 hours, 72 hours, 84 hours, 96 hours, 108 hours, 120 hours or more, preferably approximately 72 hours.

In a particular embodiment, the substrate comprising chitin and the polypeptide of the invention, preferably the polypeptide comprising or consisting of SEQ ID NO: 1, are contacted in a water solution at a temperature of approximately 50° C., during approximately 72 hours in agitation.

In a particular embodiment, the substrate comprising chitin and the polypeptide of the invention, preferably the polypeptide comprising or consisting of SEQ ID NO: 1, are contacted under agitation.

Once the above period of time is completed, that is, once the desired crystalline nanochitin is obtained, the reaction can be stopped, for example, by decreasing the temperature of the reaction mixture to a temperature at which no hydrolysis is performed, for example, by incubation on ice.

Once the above period of time is completed, that is, once the desired crystalline nanochitin is obtained, the reaction mixture can be subjected to sonication.

In a particular embodiment, the resulting crystalline nanochitin can be isolated from the rest of the components of the reaction mixture, for example, chitin that has not been cleaved, enzyme, or other products of the reaction, such as sugars and chitin nanofibers. In a more particular embodiment, the crystalline nanochitin is isolated by filtration and/or centrifugation and/or any other suitable technique known by the skilled person. In a particular embodiment, the crystalline nanochitin is isolated by filtration. In another particular embodiment, the crystalline nanochitin is isolated by centrifugation. In another particular embodiment, the crystalline nanochitin is isolated by filtration and centrifugation.

The filtration can be performed by using microfiltration, a rotatory drum filter or using a nylon filter of 5 to 20 μm mesh. To reach the maximum recovery of cellulose nanocrystals, the retentate can be resuspended in a lower volume of water and washed several times, repeating the filtration until the filtrate is clear. The chitin nanocrystals can be recovered from the filtrate and washings by centrifugation at 15000 g (relative centrifuge force) for 30 minutes to 1 hour.

The isolation by centrifugation can be performed in two steps, a first step to wash the undigested fibers, recovering the nanocrystalline chitin from the supernatant of successive washings with decreasing volumes of water followed by centrifugation at 4000 g (relative centrifuge force) for 5 minutes. The washings should be repeated suspending the sediment in water until the supernatant is clear and free of nanochitin material. The second recovery step concentrates the suspended material of the supernatant of previous washings by higher speed centrifugation at 15000 g for 30 minutes to 1 hour. After centrifugation, the sediment comprising the crystalline nanochitin can be dried, for example by spray drying or Iyophilisation.

The obtained crystalline nanochitin can be concentrated, for example by ultracentrifugation at 33000 G for a period of time of 30 minutes.

In a particular embodiment, the second method of the invention does not comprise any step directed to the chemical acid hydrolysis of the chitin, and in particular, it does not comprise treating the chitin with hydrochloric acid. In a particular embodiment, the second method of the invention does not comprise any step directed to the chemical oxidation if the chitin, and in particular, it does not comprise treating the chitin with 2, 2, 6, 6-tetramethylpiperidine-1-oxyl (TEMPO).

In a particular embodiment, the polypeptide of the invention, preferably the polypeptide comprising or consisting of SEQ ID NO: 1, is immobilized in a solid support. Non-limiting exemplary solid supports include polymers (such as agarose, sepharose, cellulose, nitrocellulose, alginate, Teflon, latex, acrylamide, nylon, plastic, polystyrene, silicone, polymethylmetacrylate (PMMA), etc.), glass, silica, ceramics, and metals. Such solid supports may take any form, such as particles (including microparticles), sheets, dip-sticks, gels, filters, membranes, microfiber strips, tubes, wells, plates (such as microplates, including 6-well plates, 24-well plates, 96-well plates, 384-well plates, etc.), fibers, capillaries, combs, pipette tips, microarray chips, etc. In some embodiments, the surface of the solid support comprises an irregular surface, such as a porous, particulate, fibrous, webbed, or sintered surface. In some embodiments, a solid support is selected from a microplate, a microarray chip, and a microparticle. In some embodiments, a solid support is at least partially composed of a polymer.

In some embodiments, the polypeptide of the invention, preferably the polypeptide comprising or consisting of SEQ ID NO: 1, is attached to a solid support through a linker moiety. In a particular embodiment, said linker comprises a protease cleavage site.

In a particular embodiment, the second method of the invention further comprises contacting the sample comprising chitin and the polypeptide of the invention, preferably the polypeptide comprising or consisting of SEQ ID NO: 1, in the presence of a reducing agent. The term “reducing agent”, has been previously defined. In a particular embodiment, the reducing agent is ascorbic acid.

The second method of the invention allows obtaining nanochitin from chitin with the polypeptide of the invention, which has LPMO activity, in the absence of an incubation step with a chitinase. Therefore, in a particular embodiment, the second method of the invention does not comprise a step of contacting the sample containing chitin with a chitinase.

### Crystalline Nanochitin

In another aspect, the invention relates to crystalline nanochitin, hereinafter crystalline nanochitin of the invention, obtained or obtainable by the second method of the invention. All the definitions, particular and preferred embodiments of the other aspects of the invention fully apply to the crystalline nanochitin of the invention.

In a particular embodiment, the crystalline nanochitin of the invention has a diameter comprised between 10 nm and 80 nm, for example between 14 nm and 75 nm, between 20 and 50 nm, between 25 nm and 40 nm, between 30 nm and 35 nm.

In a particular embodiment, the crystalline nanochitin of the invention has a length comprised between 50 nm and 2000 nm, for example, between 90 nm and 1800 nm, between 150 nm and 1500 nm, between 300 nm and 1000 nm, between 350 nm and 750 nm, preferably between 400 nm and 500 nm.

In a particular embodiment, the crystalline nanochitin of the invention has a length to width aspect ratio comprised between 5 and 50, for example between 10 and 40, for example between 12 and 30, preferably between 14 and 20.

The diameter and length of the crystalline nanochitin can be calculated using any suitable technique known by the skilled person, for example, by atomic force microscopy, as detailed in the examples herein included. Atomic force microscopy (ATM) is a technique that uses the interaction between the tip and the sample attractive-repulsion forces to create a deflection in the tip permitting to infer the imagines by mapping the deflections in each point of the sample.

In a particular embodiment, the crystalline nanochitin of the invention has a crystallinity index between 88 and 90%, preferably approximately 89%.

The crystallinity index can be determined by any suitable technique known by the skilled person, for example, X-Ray diffraction (XRD) as detailed in the examples herein included. X-Ray diffraction is a technique based on the constructive interference of monochromatic X-rays and a crystalline sample.

From the diffractograms, the crystallinity index (Cl %) can be calculated using the Segal equation:

$$Crystallinity index ( \% ) = ( {I}_{110} - {I}_{a m} ) / {I}_{110} \times 100$$

where I₁₁₀ is the intensity of the chitin crystalline peak and Iₐₘ is the intensity of the amorphous peak.

In a particular embodiment, the crystalline nanochitin of the invention as a crystallite size comprised between 4.9 nm and 6.3 nm, for example between 5.1 and 6.1 nm, between 5.3 nm and 5.9 nm, between 5.6 nm and 5.7 nm, preferably about 5.8 nm.

The crystallite size can be determined by any suitable method known by the skilled person, for example, by X-Ray diffraction as detailed in the examples herein included. The crystallite size can be measured from the diffractograms using the Scherrer's equation:

$$β = κλ / τ cos θ \times 100$$

where λ is the wavelength of the incident X-ray, θ is the angle of the (200) plane, β is the full-width at half maximum of the (200) peak, T is the crystallite size and κ is a constant value.

Without wanting to be bound to any particular theory, it is thought that when LPMO is used for obtaining crystalline nanochitin from chitin, carboxyl groups are incorporated into the chitin structure. Therefore, the nanochitin of the invention differs from the crystalline nanochitin obtained by acid hydrolysis or by oxidation with TEMPO in that it includes carboxyl groups. The term “carboxyl groups”, as used herein refers to a —COOH group. An optionally substituted carboxyl includes, for example, a —COOR group, wherein R is H or any substituent group.

### Composite Material and Method for its Preparation

In another aspect the invention refers to a composite material comprising:
(a) crystalline nanochitin, wherein said crystalline nanochitin is obtained by a method comprising contacting a sample comprising chitin with a copper-bound polypeptide having LPMO activity under suitable conditions for the oxidative cleavage of chitin; and
(b) a carbon material selected from the group consisting of carbon nanotubes, carbon nanofibers, graphene, graphene oxide and a mixture thereof.

The term “crystalline nanochitin” has been previously defined. All the particular and preferred embodiments, regarding this term in connection with the method for producing crystalline nanochitin also apply to the composite material of the invention.

In a particular embodiment, the crystalline nanochitin has a diameter comprised between 10 nm and 80 nm, for example between 14 nm and 75 nm, between 20 and 50 nm, between 25 nm and 40 nm, between 30 nm and 35 nm.

In a particular embodiment, the crystalline nanochitin has a length comprised between 50 nm and 2000 nm, for example, between 90 nm and 1800 nm, between 150 nm and 1500 nm, between 300 nm and 1000 nm, between 350 nm and 750 nm, preferably between 400 nm and 500 nm.

In a particular embodiment, the crystalline nanochitin has a length to width aspect ratio comprised between 5 and 50, for example between 10 and 40, for example between 12 and 30, preferably between 14 and 20.

In a particular embodiment, the crystalline nanochitin has a crystallinity index between 88 and 90%, preferably approximately 89%.

In a particular embodiment, the crystalline nanochitin has a crystallite size comprised between 4.9 nm and 6.3 nm, for example between 5.1 and 6.1 nm, between 5.3 nm and 5.9 nm, between 5.6 nm and 5.7 nm, preferably about 5.8 nm.

The crystalline nanochitin comprised in the composite material of the invention is obtained by a method comprising contacting a sample comprising chitin with a copper-bound polypeptide having LPMO activity under suitable conditions for the oxidative cleavage of chitin. The terms “samples comprising chitin”, “copper-bound” and “suitable conditions for the oxidative cleavage of chitin” has been previously defined in the context of the second method of the invention.

The term “polypeptide having LPMO activity”, as used herein, refers to a lytic polysaccharide monooxygenase enzyme or LPMO. The term “LPMO” has been previously defined. Illustrative non-limitative examples of LPMOs that can be used for obtaining the crystalline nanochitin that forms part of the composite material of the invention includes the LPMO of Table 1.

Without wanting to be bound to any particular theory, it is thought that when LPMO is used for obtaining crystalline nanochitin from chitin, carboxyl groups are incorporated into the chitin structure. Therefore, in a particular embodiment, the composite of the invention comprises crystalline that includes carboxyl groups.

In a particular embodiment, the polypeptide having LPMO activity is the polypeptide of the invention, preferably the polypeptide comprising or consisting of SEQ ID NO: 1. In this particular embodiment, the crystalline nanochitin is the crystalline nanochitin of the invention, that is, a crystalline nanochitin that is obtainable by the method of the invention

Component (b) of the composite material of the invention is a carbon material selected from the group consisting of carbon nanotubes, carbon nanofibers, graphene, graphene oxide and a mixture thereof.

In the context of the present invention, the term “carbon material” is to be interpreted, unless stated otherwise, as a group consisting of carbon nanotubes, carbon nanofibers, graphene, graphene oxide and a mixture thereof. These materials have remarkable electrical conductivity, tensile strength and thermal conductivity. The term “carbon nanofibers” is synonym to “vapor grown carbon fibers” or “vapor grown carbon nanofibers” and refers to cylindrical nanostructures (at least one dimension in the nanometer range) with graphene layers arranged as stacked cones, cups or plates. “Carbon nanofibers” differ from what is termed as “carbon nanotubes” in that carbon nanotubes are characterized by graphene layers wrapped into perfect cylinders. Carbon nanotubes encompasses single-wall carbon nanotubes (SWCNTs) with diameters in the range of a nanometer but can also refer to multi-wall carbon nanotubes (MWCNTs) consisting of nested single-wall carbon nanotubes. The carbon nanotube may be one or more selected from the group consisting of single-walled, double-walled, thin multi-walled, multi-walled, and roped types. The term “graphene” is to be interpreted as an allotrope of carbon in the form of a single layer of atoms in a two-dimensional hexagonal lattice, characterized by tightly packed carbon atoms and a sp² orbital hybridization. Graphene is a zero-gap semiconductor and displays remarkable electron mobility at room temperature, with reported values in excess of 15000 cm²×V⁻¹×s⁻¹. The term “graphene oxide” refers to the oxidized form of graphene, and it can be obtainable by techniques known to the skilled person. For example, through chemical modification of graphite in acidic medium. Graphene oxide displays higher solubility when compared to that of graphene.

In a particular embodiment, the carbon material is graphene oxide.

In a particular embodiment, the composite material of the invention comprises (a) crystalline nanochitin obtained by the method of the invention comprising using the polypeptide of the invention, preferably the polypeptide comprising or consisting of the sequence of SEQ ID NO: 1, and (b) graphene oxide.

In a particular embodiment, the composite material of the invention is an electrically conductive composite material.

In the context of the present invention, “electrically conductive composite material” refers to a material as an object or type of material that allows the flow of charge (electrical current) in one or more directions. Electrical current is generated by the flow of negatively charged electrons, positively charged holes, and positive or negative ions in some cases. Therefore, in a particular embodiment of the invention, the cellulose composite material of the invention is capable of conducting electricity, i.e., has electrical conductivity, or simply conductivity. Electrical conductivity (or conductivity, measured as Siemens per meter, S/m) is a fundamental property of a material that quantifies how strongly it conducts electric current.

In a particular embodiment of the invention, the composite material is characterized by a conductivity value equal to or greater than 11 S/m, measured by the electrical impedance four-terminal sensing technique. In a more particular embodiment, when the composite material of the invention comprises 10% weight of reduced graphene oxide, the composite material is characterized by a conductivity value equal to or greater than 11 S/m, measured by the electrical impedance four-terminal sensing technique.

In a particular embodiment of the invention, the crystalline nanochitin and the carbon material are homogeneously distributed within the composite material. Preferably, the composite material comprises at least 0.1%, at least 0.2%, at least 0.3%, at least 0.4%, at least 0.5%, at least 0.6%, at least 0.8%, at least 1%, at least 2%, at least 5%, at least 10%, at least 15%, at least 20%, at least 25% or at least 30% w/w of carbon material, with regards to the sum of the weights of the crystalline nanochitin and said carbon material.

In another aspect, the invention relates to a method for the preparation of the composite material of the invention, comprising the steps of:
(i) preparing a suspension comprising crystalline nanochitin and a carbon material selected from the group consisting of carbon nanotubes, carbon nanofibers, graphene, graphene oxide and a mixture thereof;
(ii) mixing the suspension; and
(iii) optionally reducing said carbon material in the mixture of the previous step.

In a particular embodiment, step (iii) comprises the reduction of graphene oxide to graphene. This step is preferably conducted in the presence of a reducer or in a reducing medium selected from the group consisting of ascorbic acid, hydrazine, ultraviolet light, and mixtures thereof. Preferably, the reducer is ascorbic acid. If necessary, the reduction is carried out at a temperature higher than 25° C. The skilled person will readily understand that the temperature must not be so high that the cellulose material is affected. In a particular embodiment, the reduction is carried out at a temperature comprised between 3° and 100° C., preferably between 8° and 95° C.

In a particular embodiment, the method further comprises drying the mixture

In a particular embodiment, the method comprises mixing a suspension comprising crystalline nanochitin and a carbon material, preferably graphene oxide, at a concentration of at least 0.1%, at least 0.2%, at least 0.3%, at least 0.4%, at least 0.5%, at least 0.6%, at least 0.8%, at least 1%, at least 2%, at least 5%, at least 10%, at least 15%, at least 20%, at least 25% or at least 30% w/w of carbon material, preferably graphene oxide, with regards to the sum of the weights of the crystalline nanochitin and said carbon material.

When the composite material of the invention is intended to be used as a bioink the material can be obtained by mixing suspension of crystalline nanochitin comprising carboxyl groups with 5% w/w of graphene oxide. The mixture can be then sonicated.

In a particular embodiment, the invention relates to a composite material obtained or obtainable by the method for the preparation of the composite material of the invention.

### Uses of the Crystalline Nanochitin and Composite Material

In another aspect, the invention relates to the use of the crystalline nanochitin of the invention or of the composite material of the invention as a bioink for three-dimensional bioprinting or as a substrate for cell growth.

The term “bioink”, as used herein, refers to any substance, whether liquid, solid or semi-solid, suitable for 3D printing technology. A bioink can comprise cells and/or a carrier material, usually a biopolymer gel, which acts as a three-dimensional molecular scaffold. Cells can attach to this scaffold, which enables them to spread, grow and proliferate. A material suitable as a bioink shall be biocompatible or biodegradable. The tem “bioink” can be used to refer to the carrier material alone, or to the combination of the carrier material and the cells.

The term “three-dimensional bioprinting” refers to a technology for producing 3D constructs from biomaterials by using 3D printing technology. For bioprinting cells and a biocompatible material interacting therewith, also called “bioink”, are placed at a specific site by stacking. Examples of constructs that can be made by this technology include artificial tissues, tissue models, functional biomaterials, biomedical devices, scaffolds and the like.

The term “substrate for cell growth” refers to a material to which cells can attach, and which allows the attached cells to spread, grow and proliferate.

### EXAMPLES

### Material and Methods

Ancestral sequence reconstruction (ASR) of LPMO. Fifty-one LPMO from auxiliary activity family 10 (AA10) were downloaded from the NCBI database. Sequences belonging to three bacterial phyla: Proteobacteria, Actinobacteria, and Firmicutes. All sequence ID numbers are listed in Table 1. Alignment of the sequences was performed using MUSCLE software on the MEGA platform and manually edited (Manuel, M., Zootaxa 2013, 3652, 453-474). The best evolutionary model was inferred using MEGA, resulting in the Jones-Tylor-Thornton (JTT) with gamma distribution model. The phylogeny was carried out using BEAST v1.8.4 package software, including the BEAGLE library for parallel processing and Bayesian inference using Markov chain Monte Carlo (MCMC). Monophyletic groups were established for Proteobacteria, Actinobacteria, and Firmicutes and JTT model with eight gamma categories and invariant distributions, Yule model for speciation, 20 million generations of length chain, and sampling every 1000 generations. The divergence times were estimated by uncorrelated log-normal clock model (UCLN), using molecular information from Time Tree Of Life (TTOL) with default birth and death rates (Hedges, S. B.; Marin, J.; Suleski, M.; Paymer, M.; Kumar, S., 2015, 32 (4), 835-845). Calculations were run in a multicore server. From the generated trees the 25% of them were discarded as burn-in with the LogCombiner utility from BEAST. MCMC log file was verified using TRACER ensuring all parameters showed effective sample size (ESS)>100. Posterior probabilities of all nodes were above 0.65, and most of them were near 1. Figure tree v1.4.2 was used to visualize and edit the phylogenetic tree. Finally, ancestral sequence reconstruction was performed by maximum likelihood using PAML 4.8 with a gamma distribution for variable replacement rates across sites and the JTT model (Yang, Z., Molecular biology and evolution 2007, 24 (8), 1586-1591). Posterior probabilities were calculated for all amino acids, and the residue with the highest posterior probability was chosen for each site. High posterior probabilities ensure the correctness of each bifurcation given the sequence collection, alignment and model used. Last Actinobacteria/Firmicutes common ancestor (LAFCA) was selected for laboratory resurrection.

TABLE 1
LPMO sequences from the species used in the construction of
the phylogenetic tree. On the left hand the NCBI ID and on
the right hand the species where the sequence belongs.

| NCBI ID | Species |
|---|---|
| WP_098855344.1 | Bacillus thuringiensis |
| WP_000742280.1 | Bacillus cereus |
| WP_097855297.1 | Bacillus toyonensis |
| WP_017149289.1 | Bacillus bingmayongensis |
| WP_033676157.1 | Bacillus gaemokensis |
| WP_084157953.1 | Bacillus manliponensis |
| WP_089967265.1 | Lihuaxuella thermophila |
| WP_006676405.1 | Paenibacillus dendritiformis |
| WP_087435284.1 | Paenibacillus apiarius |
| WP_087445137.1 | Paenibacillus thiaminolyticus |
| WP_036222403.1 | Lysinibacillus sphaericus |
| WP_082332692.1 | Lysinibacillus contaminans |
| WP_036094696.1 | Listeria newyorkensis |
| WP_070239340.1 | Listeria monocytogenes |
| WP_010772090.1 | Enterococcus caccae |
| WP_069664391.1 | Enterococcus termitis |
| WP_034688872.1 | Enterococcus mundtii |
| WP_076633771.1 | Lactobacillus plantarum |
| WP_021731441.1 | Lactobacillus |
| WP_055538846.1 | Streptomyces neyagawaensis |
| WP_055712380.1 | Streptomyces torulosus |
| WP_005486591.1 | Streptomyces bottropensis |
| WP_033531196.1 | Streptomyces galbus |
| WP_046731057.1 | Streptomyces humi |
| WP_093654234.1 | Streptomyces wuyuanensis |
| WP_026416933.1 | Actinomadura oligospora |
| WP_017615731.1 | Nocardiopsis salina |
| WP_039991854.1 | Serratia odorifera |
| WP_044552811.1 | Serratia liquefaciens |
| WP_054307289.1 | Serratia rubidaea |
| WP_085118064.1 | Serratia proteamaculans |
| WP_050539174.1 | Yersinia mollaretii |
| WP_051714045.1 | Shewanella |
| WP_099457584.1 | Shewanella xiamenensis |
| WP_009837614.1 | Pseudoalteromonas tunicata |
| WP_082916329.1 | Pseudoalteromonas neustonica |
| WP_072034004.1 | Rahnella aquatilis |
| WP_042289422.1 | Citrobacter |
| WP_077227974.1 | Leclercia adecarboxylata |
| WP_045353104.1 | Enterobacter cloacae complex |
| WP_059288204.1 | Enterobacter hormaechei |
| WP_053064552.1 | Klebsiella oxytoca |
| WP_064352001.1 | Klebsiella |
| WP_084204703.1 | Aeromonas popoffii |
| WP_079516857.1 | Kosakonia oryzae |
| WP_062702299.1 | Chryseobacterium indologenes |
| WP_084694528.1 | Chryseobacterium vrystaatense |
| WP_090024402.1 | Chryseobacterium oleae |
| WP_082798703.1 | Chryseobacterium cucumeris |
| WP_089695525.1 | Chryseobacterium culicis |
| WP_083997416.1 | Chryseobacterium angstadtii |

Protein expression and purification. Previously described protocol for LPMO expression and purification was used (Rodrigues, K. B.; Macêdo, J. K.; Teixeira, T.; Barros, J. S.; Araújo, A. C.; Santos, F. P.; Quirino, B. F.; Brasil, B. S.; Salum, T. F.; Abdelnur, P. V., Carbohydrate research 2017, 448, 175-181). Genes encoding the ancestral and extant LPMO proteins were synthesized, and codon optimized for expression in E. coli cells (Life Technologies). The genes were cloned into pET28a vector and transformed onto E. coli BL21 cells (DE3) (Life Technologies) for protein expression (Kim, E. Y.; Jakobson, C. M.; Tullman-Ercek, D., PloS one 2014, 9 (11), e113814). Bacteria were incubated in LB medium at 37° C. until OD₆₀₀ reached 0.6; IPTG was added to the medium to 1 mM concentration for protein induction overnight at 30° C. After centrifugation, cell pellets were resuspended in extraction buffer (50 mM sodium phosphate, pH 7.0, 300 mM NaCl), incubated with lysozyme for 30 min and mechanically lysed using French press or ultrasound. Cell debris was separated by ultracentrifugation at 33,000×g for 1 h. For purification, the supernatants were mixed with His GraviTrap affinity column (GE Healthcare) and eluted with elution buffer containing imidazole (50 mM sodium phosphate, pH 7.0, 300 mM NaCl, 150 mM imidazole) (Gardner, J. G.; Crouch, L.; Labourel, A.; Forsberg, Z.; Bukhman, Y. V.; Vaaje-Kolstad, G.; Gilbert, H. J.; Keating, D. H., Molecular microbiology 2014, 94 (5), 1121-1133). The proteins were copper saturated by incubation with Cu_{(II)}SO₄ at a ratio of 1:3 for 30 min at room temperature (Stepnov, A. A.; Forsberg, Z.; Sorlie, M.; Nguyen, G.-S.; Wentzel, A.; Røhr, Å. K.; Eijsink, V. G. H., 2021, 14 (1), 28). The proteins were then further purified and the non binded copper was removed by size exclusion chromatography using a Superdex 200HR column (GE Healthcare). The buffer used was 50 mM sodium phosphate pH 7.0. The purified proteins were finally verified by SDS-PAGE with 12% acrylamide gels. The protein concentration was calculated by measuring the absorbance at 280 nm in Nanodrop 2000C, using the equation ε₂₈₀ of x M⁻¹ cm⁻¹ and MW of x g mol⁻¹, with the theoretical extinction coefficient of 39.545 and molecular mass of 20 kDa.

LPMO characterization. Reactions were performed in 96-well plates mixing 166 μL of 100 mM phosphate buffer pH 8, 20 μL of 10 mM DMP (final concentration of 1 mM) (Breslmayr, E.; Haniek, M.; Hanrahan, A.; Leitner, C.; Kittl, R.; Šantek, B.; Oostenbrink, C.; Ludwig, R., Biotechnology for biofuels 2018, 11 (1), 1-13), and 4 μL of 5 mM H₂O₂ (final concentration of 0.1 mM). Finally, 10 μL of enzyme dilution were added at a suitable concentration to each well in a final volume of 200 μL per well. Plates were orbitally mixed, and the reaction was followed at 50° C. Activity was calculated by measuring the increase of absorbance at 469 nm for 5 minutes using the molar absorption coefficient of coerulignone (E469=53,200 M⁻¹ cm⁻¹) to calculate the peroxidase activity of LPMO. For thermal stability assay, the LPMO enzymes were preincubated to the targeted temperatures for 5 minutes in a 1.5 mL Eppendorf in a thermo mixer and then place them in ice for 5 minutes. The reaction was carried out at a 96 well plate (Thermo Fisher) at 50° C. 169.5 μL 100 mM phosphate buffer pH 8, 20 μL 10 mM DMP as a chromogenic substrate that makes a final concentration of 1 mM, 0.5 μL of 5 mM H₂O₂, and 10 μL of enzyme dissolution with adequate enzyme concentration making a final volume of 200 μL. Absorbance at 469 nm was measured for 30 minutes at Epoch 2 spectrophotometer. For pH assay, the pH of the reaction was the pH of the buffer (169.5 μL out of 200 μL). Different pH buffer dissolutions were prepared from pH 3 to pH 10: 100 mM citric acid for pH 3-5, 100 mM phosphate buffer for pH 6-7, 100 mM carbonate buffer for pH 8 100 mM Boric acid for 9-10.

Nanochitin enzymatic isolation. Commercial α-Chitin from Sigma Aldrich was used as substrate. 2.5% α-Chitin dissolution in water was used with a ratio of 10 mg on each enzyme (LFACA LPMO and Bt LPMO) per gram of substrate. As reductant, 2 mM ascorbic acid from Sigma Aldrich was used. The oxidative cleavage was carried out at 50° C. in agitation for 72 hours. Reactions were stopped by placing them on ice, and the mixtures were sonicated with a microtip sonicator UPH 100H Ultrasonic Processor (Hielscher) for 25 min at 75%. Nanochitin was isolated by several centrifugation steps and concentrated by ultracentrifugation at 33000 G for 30 minutes. Pellets were resuspended in water or 2% acetic acid and lyophilized in a Telstar Lyoquest for physical and chemical characterization by freeze-drying for 24 hours. Nanochitin produced by hydrochloric acid form was donated from “Material and Technology group (GMT)” from UPV/EHU.

Nanochitin EnCNCh/graphene films. Nanochitin films were prepared by casting method with an EnCNCh suspension of 1 wt % sonicated for 1 hour. The suspension was placed in a Teflon mold and dried for 2 hours at 50° C. Conductive films were fabricated by EnCNCh suspension and with graphene oxide at different final concentrations: 0.3, 1, 2, 5, 10, and 15% wt of GO. Graphene oxide (4 wt % water suspension) was kindly supplied by Graphenea (San Sebastian, Spain). The mixture was sonicated in a sonicator bath to assure homogeneity and cast with the help of a vacuum bomb using an Ultrafiltration disc of 30 KDa from Merc. The wet film was dried at 50° C. for 2 hours, obtaining films with a thickness of around 50 m. GO in the nanopapers was reduced by placing the films in ascorbic acid solution (30 mg/mL) for 2 hours at 95° C. and then washed using miliQ water before drying the films at 50° C. for 2 hours.

Nanochitin EnCNCh/graphene bioinks. Bioinks for 3D printing were prepared 10% wt EnCNCh suspension and with 5% wt graphene oxide. The mixture was sonicated in a sonicator bath to assure homogeneity. After printing and freeze-drying the scaffolds, GO in the scaffold was reduced by placing the films in ascorbic acid solution (30 mg/mL) for 2 hours at 95° C. and then washed using miliQ water before drying the films at 50° C. for 2 hours.

Characterization of materials. Fourier transform infrared (FTIR) spectroscopy spectra were recorded in attenuated reflection (ATR) mode to analyze functional groups of chitin, EnCNCh, and acid CNCh. A Perkin-Elmer Frontier FTIR spectrophotometer was used, equipped with an ATR sampling stage within the wavenumber of 4000 and 650 cm⁻¹, with 32 scans and a resolution of 4 cm⁻¹.

Atomic Force Microscopy (AFM) was used to study CNCh morphology of the nanoparticles. A Nanoscope V scanning probe microscope (Multimode 8 Bruker Digital instruments) was used, using an integrated force generated by cantilever/silicon probes. Images were obtained at room temperature, in taping mode, applying 320 kHz resonance frequency and 5-10 nm tip radius and 125 μm long. Sample preparation was made by spin coating using a Spincoater P6700 at 200 rpm for 60 seconds on mica substrate. AFM height and phase images were collected simultaneously in all the samples. The size of the images was 3×3 μm, and fibers of different size were distinguished. The length and diameter of 100 nanoparticles was measured to calculate the average length, diameter, and aspect ratio (Length/Diameter).

Crystalline structure, crystallinity, and crystallite size of nanochitin was studied using X-ray diffraction (XRD) powder diffraction patterns. Data were collected at room temperature using a Philips X'pert PRO automatic diffractometer from 5 to 50° and a PIXcel solid state detector (active length in 2θ 3.347°) operating at 40 kV and 40 mA, in theta configuration, a secondary monocromator with Cu-Kα radiation (λ=1.5418 Å) and a PIXcel solid state detector (active length in 2θ 3.347°). An antiscattering slit and a fixed divergence giving a constant volume of sample illumination were used. From the diffractograms, the crystallinity index (Cl %) was calculated by Segal equation (Segal, L.; Creely, J.; Martin Jr, A.; Conrad, C., Textile research journal 1959, 29 (10), 786-794):

$$Crystallinity index ( \% ) = \frac{( {I}_{110} - {I}_{a m} )}{{I}_{110}} \times 100$$

where I₁₁₀ is the intensity of the cellulose crystalline peak and lam is the intensity of the amorphous peak. Crystallite size was measured using the Scherrer's equation:

$$β = \frac{κ λ}{τ cos θ} \times 100$$

where λ is the wavelength of the incident X-ray, θ is the angle of the (110) plane, β is the full width at half maximum of the (110) peak, T is the crystallite size, and κ is a constant value.

To study nanochitin structure, solid-state cross-polarization magic angle spinning 13C nuclear magnetic resonance (¹³C CP/MAS NMR) was used. ¹³C CP/MAS NMR spectra were measured using a 400 MHz BRUKER system equipped with a 4 mm MASDVT TRIPLE Resonance HYX MAS probe. 2K scans were taken at Larmor frequencies of 400.17 MHz and 100.63 MHz for ¹H and ¹³C nuclei. Chemical shifts were reported relative to the signals of 13C nuclei in glycine. Sample rotation frequency was 12 kHz, and relaxation delay was 5 s. Polarization transfer was achieved with RAMP cross-polarization (ramp on the proton channel) with a contact time of 5 ms. High-power SPINAL 64 heteronuclear proton decoupling was applied during acquisition.

Thermogravimetric analysis (TGA) was used to study the thermal stability of nanochitin and hybrid nanopapers. The data was recorded using TGA/SDTA 851 Mettler Toledo equipment, where 10 mg of the samples were heated from 30 to 800° C. in a nitrogen atmosphere with a scanning rate of 10° C./min. The initial degradation temperature (To) is described as the loss of 5% of the weight of the total sample and the maximum degradation temperature (Td) is the minimum of the degradation peak in the derivative of thermogravimetric curves (DTG).

The morphology of the nanopaper surface and graphene was analyzed by SEM using a FEI ESEM Quanta 200 microscope operating at 5-20 kV. Nanopapers were put on carbon tape for adhesion.

Electrical conductivity was measured by a four-point probe method using a Probe Station 4 Everbeing. The specific resistances (q) were calculated with the sheet resistances (Rs, Ω/squares) and the thickness of the nanopapers (t, cm) in Eq.:

$$q = {R}_{s} \times t$$

The specific resistance was used calculated to infer the corresponded conductivity (S/cm) with the following Eq. that was transformed to S/m.

$$r = 1 / q$$

The percolation threshold, ρc, was measured using a power-law equation based on the percolation theory (Zhu, C. et al., ACS nano 2018, 12 (7), 7028-7038):

$$σ = {σ}_{f} \times {( ρ - {ρ}_{c} )}^{n}$$

where σ is the conductivity of the nanopaper, ρ_{f} is the rGO conductivity, ρ is the rGO content expressed at volume fraction, and n is the exponent describing the rapid variation of the conductivity near the percolation threshold (ρ_{c}).

3D printing of EnCNCh-based scaffold. Scaffolds were printed using a Voladora 3D printer (Tumaker, S. L. Spain) which has been modified for layer by layer syringe extrusion 3D printing. Honeycomb like scaffold and button like scaffolds were printed directly on poly-tetrafluoroethylene slides at room temperature using a needle of 0.8 mm in diameter and speed of 5 mm/s.

2D and 3D cell cultures. Cells were maintained in DMEN+10% FBS medium supplemented with 1% (w/v) L-glutamine and penicillin-streptomycin (100 IU/ml). HEK293T cells were a kind gift of Dr. Maria Munoz Caffarel. Before growing cells in the material, EnCNCh film and EnCNCh scaffold were sterilized by UV for 3 hours. 20,000 cells were seeded on each substrate and let to growth for 3 days in the incubator at 37° C. in 5% CO₂. Cells were fixed in 4% formaldehyde for 30 min and washed with PBS. Films were dehydrated by increasing concentration of ethanol solution until 100% concentration of ethanol was reached. After fixation Cells were dyed with DAPI solution (5 mg/ml) and CellMask Orange Plasma membrane Stain (5 mg/mL) diluted in PBS (1:2000) and after washing each sample three times with PBS. DAPI-Orange Plasma-stained cells were observed after 20 min of incubation by confocal microscopy.

### Results and Discussion

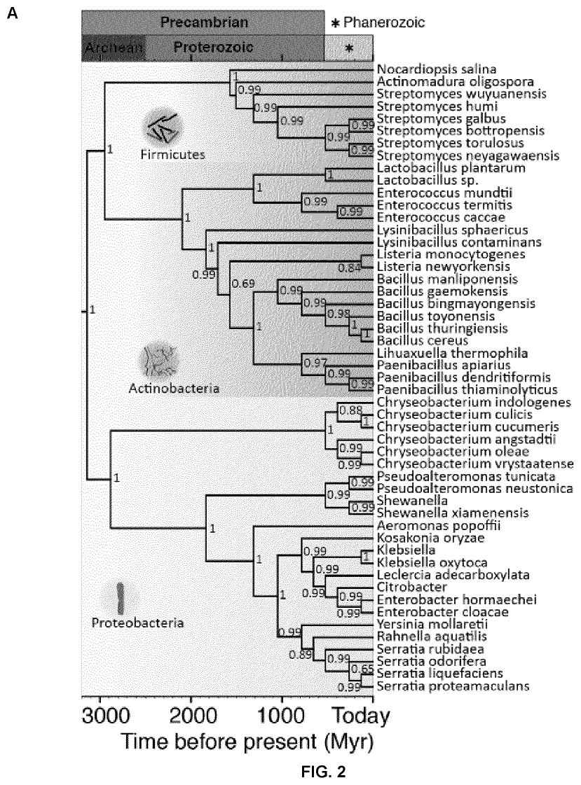

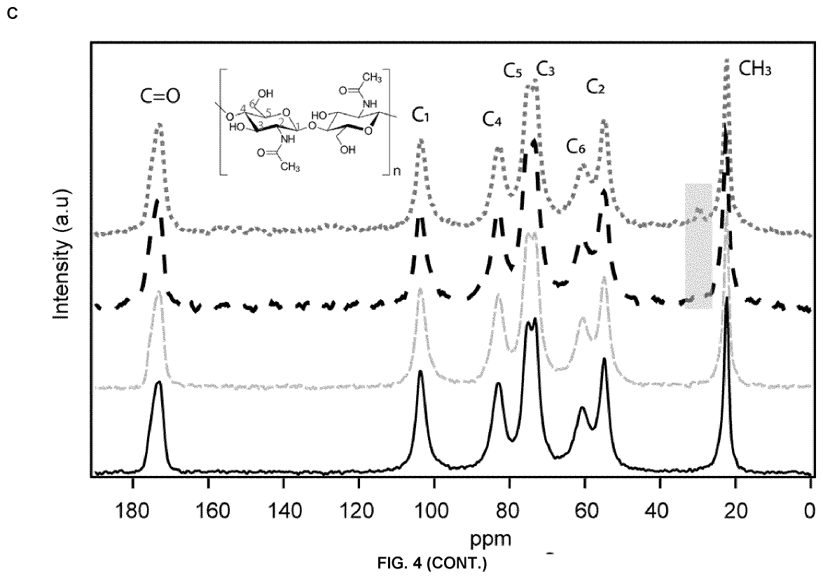

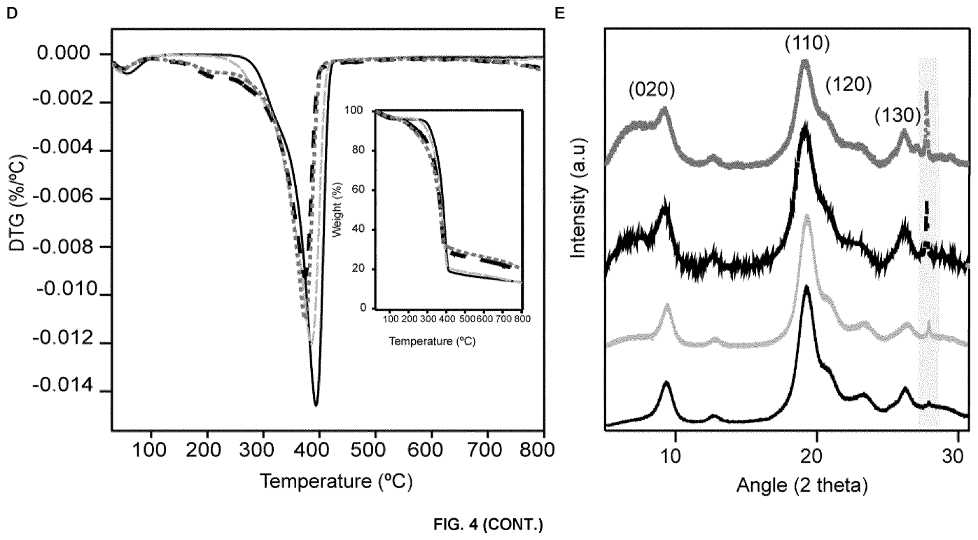

To infer the sequences of extinct bacterial LPMOs, the sequence of 51 extanct sequences of the AA10 LPMO family were first retrieved from UNIPROT database using as query sequence Bt LPMO (Table 1), which has been described to act on chitin. A diverse collection of sequences from Actinobacteria, Firmicutes, and Proteobacteria phyla was obtained, which diverged more than 3By ago. This distribution indicates that LPMO were likely present already in an organism that lived in the Archean eon. This is consistent with the idea of the ancient origin of chitinolytic enzymes in bacteria. The 51 sequences were aligned, resulting in a compact, well-resolved alignment with significant conserved portions. Using the alignment, a phylogenetic chronogram was obtained using Bayesian inference (Randall, R. N. et al., Nature communications 2016, 7 (1), 1-6), in which the three clades were well resolved (FIG. 2a). The tree was dated using information from the Time Tree Of Life (TTOL) (Hedges, S. B. et al., Molecular biology and evolution 2015, 32 (4), 835-845). Using both alignment and phylogeny, the most probable ancestral sequence for each node using maximum likelihood was inferred (Drummond, A. J. et al., Molecular biology and evolution 2012, 29 (8), 1969-1973; Xu, B. and Yang, Z., Molecular biology and evolution 2013, 30 (12), 2723-2724) resulting posterior probabilities ranging from 0.87 to 0.99 (FIG. 7). The oldest node corresponding to the Last Firmicutes/Actinobacteria Common Ancestor (LFACA), which lived ˜3 billion years ago, was selected for laboratory resurrection (FIG. 2a). LFACA LPMO shares 63% identity with Bt LPMO. The gene encoding LFACA LPMO was synthesized, cloned, and expressed in E. coli strains (FIG. 8). Ancestral LPMO express at higher concentration than Bt LPMO, around 25% more (FIG. 8), a common feature of ancestral enzymes.

LPMO catalyzes the oxidative depolymerization of carbohydrates such as cellulose and chitin. However, LPMO activity can be readily assayed by a colorimetric assay using 2,6-dimethoxyphenol (2,6-DMP) and H₂O₂ as substrate and co-substrate, respectively (Breslmayr, E. et al., Biotechnology for biofuels 2018, 11 (1), 1-13), releasing the product coerolignone, with stoichiometry 1:1. Following this test, thermal stability of LFACA LPMO in the range of 30-90° C. was determined and compared with the activity of Bt LPMO. As shown in FIG. 2b, LFACA LPMO demonstrates higher activity at all temperatures, and the difference is especially significant when incubated at higher temperatures in the range 50-80° C. with maximum performance at 70° C. for both. Similarly, the activity of LFACA LPMO is higher than that of Bt LPMO in the pH range 3-10, especially above 5 (FIG. 2c). Higher thermal and pH performance is a common trait of ancestral enzymes.

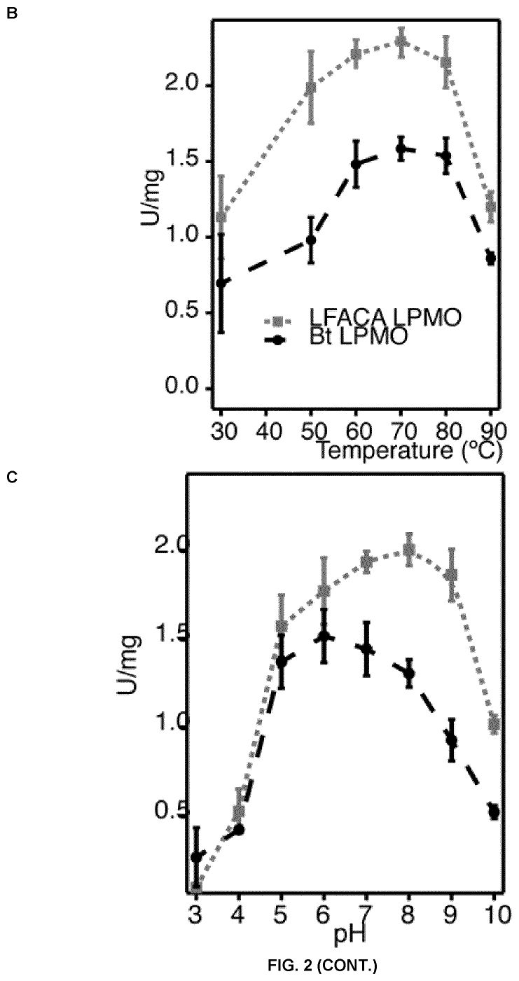

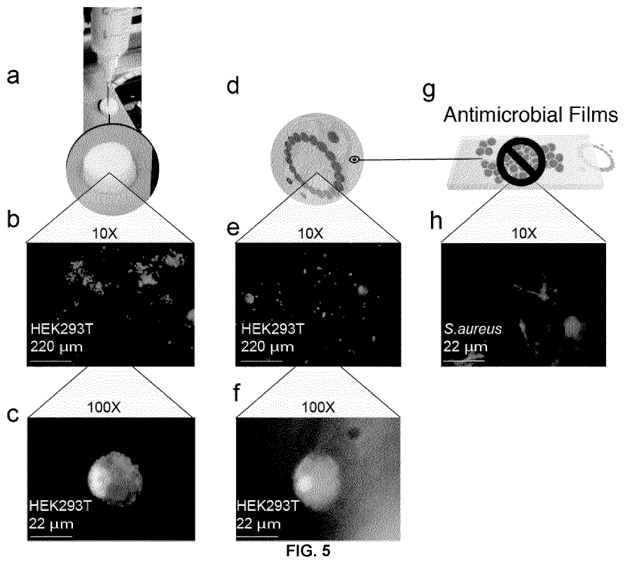

Given the ability of LPMO from family AA10 to depolymerize chitin, the efficiency of LFACA LPMO to generate nanochitin was tested and compared with extant Bt LPMO. α-Chitin flakes with no previous treatment were used as a substrate, incubated with both LPMO enzymes measuring the nanochitin mass produced at different times. As shown in FIG. 2d, nanochitin was obtained at a much higher rate when using the ancestral LFACA LPMO, i.e., 4 mg/72 h for LFACA EnCNCh versus 1.8 mg/72 h for Bt EnCNCh, at 72 hours of oxidative cleavage, the narrowest size distribution was obtained (FIG. 9). The morphology of these nanochitin products was analyzed using atomic force microscopy and also compared with crystalline nanochitin obtained by acid treatment (AcCNCh). As shown in FIG. 3a, CNChs are obtained after 72 h oxidative cleavage with both LPMOs. LFACA EnCNCh displays an average length of 458 nm and diameter of 32.3 nm, whereas Bt EnCNCh features 502 nm in length and 35.5 nm in diameter (FIG. 3b). These proportions contrast with AcCNCh size of 178.6 nm and 15.0 nm of length and diameter (FIG. 3b and Table 2). EnCNCh with both enzymes mostly shows fiber-like shapes preserving chitin natural and hierarchical structure, unlike acid treatment that destroys amorphous parts not preserving its native structure. Longer oxidative cleavage periods (96 h) seem to agglomerate CNCh through hydrogen bonds and Wand der Waals forces as previously reported for crystalline nanocellulose.

TABLE 2
Average length and diameter and L/D aspect ratio of EnCNCh particles
obtained from α-chitin hydrolysis with LFACA LPMO and extant
Bt LPMO at times of 24 hours, 48 hours, 72 hours, and 96 hours,
as well as CNCh obtained by hydrochloric acid hydrolysis. Average
parameters and S.D. were calculated with the length and diameters
of 100 particles for each condition.

| | Time | Length | Diameter | |
|---|---|---|---|---|
| Enzyme | (Hours) | (nm) | (nm) | L/D |
| LFACA LPMO | 24 | 1258.2 ± 823.2 | 58.1 ± 22.3 | 21.7 |
| (EnCNCh) | 48 | 850.3 ± 562.3 | 52.3 ± 18.8 | 16.4 |
| | 72 | 458.3 ± 365.0 | 32.3 ± 18.2 | 14.2 |
| | 96 | 975.6 ± 804.5 | 48.3 ± 22.1 | 20.1 |
| Bt LPMO | 24 | 1334.2 ± 863.5 | 62.0 ± 23.6 | 21.5 |
| (EnCNCh) | 48 | 968.5 ± 496.3 | 57.0 ± 14.2 | 16.9 |
| | 72 | 502.6 ± 215.1 | 35.2 ± 1 = 3 | 14.3 |
| | 96 | 1058.3 ± 65.3 | 51.7 ± 22.5 | 20.5 |
| Ac CNCh | — | 178.6 ± 67.2 | 15.8 ± 8.3 | 11.3 |

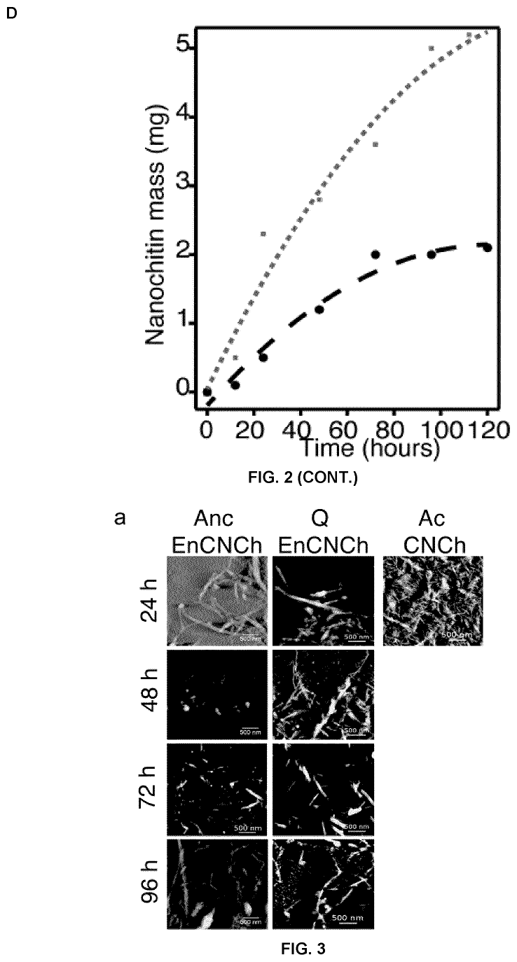

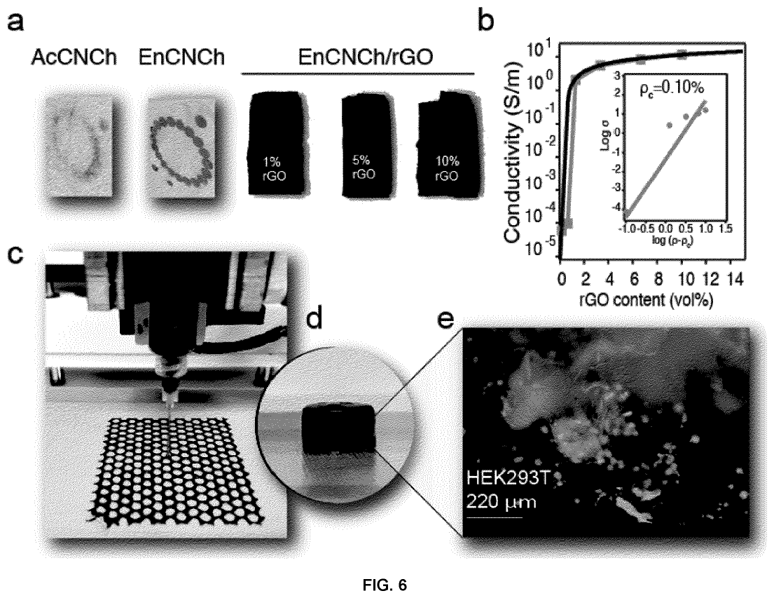

To further analyze the chemical modifications made by the LPMO enzymatic oxidative cleavage on chitin, FTIR technique (FIG. 4a) was used. After 72 h treatment, LPMO oxidative cleavage was found to lead to an oxidized EnCNCh, using either ancestral or modern LPMO. This is demonstrated by a new peak at 1710 cm⁻¹ (FIG. 4b) that corresponds to C═O stretching of COOH groups. Also, the peak intensity at 1621 cm⁻¹ increases, corresponding to C═O vibration region for carboxylic acid from amide I CO. These peaks were not observed in AcCNCh spectrum and are similar to those present in TEMPO-oxidized α-chitin at 1740 cm⁻¹, corresponding to free carboxyl groups. The absorption band around 1740 cm⁻¹ increases with reaction time (FIGS. 10 & 11). Furthermore, chemical analysis by CP/MAS ¹³C NMR proved that EnCNCh maintained native chitin structure. Additionally, the oxidative cleavage is demonstrated by a shoulder at 178 ppm from carboxyl groups. A change in the peak of C₆ at 66 ppm for EnCNCh related to COOH was observed. Another new chemical signature in enzymatic CNCh is demonstrated by a peak at 25 ppm at CP/MAS ¹³C NMR spectra (FIG. 4c) that it is hypothesized to be a methyl group oxidized to CH₂O-TEMPO-oxidized chitin or chitin nanocrystals do not show this peak, suggesting that LPMO mediated oxidation goes beyond TEMPO oxidation. Nevertheless, a slight effect on thermal stability by LPMO oxidative cleavage was observed; EnCNCh showed a degradation temperature of 374° C., which is slightly lower than that of recalcitrant α-chitin at 394° C. and AcCNCh at 385° C. (FIG. 4d and Table 3). Finally, using XRD analysis, EnCNCh was found to maintain native crystal structure with a crystallinity index (Cl) around 90%, higher than α-chitin (FIG. 4e and Table 4).

TABLE 3
Onset degradation temperature (To), maximum thermal degradation
temperature (Td) and char residue (%) of α-chitin,
LFACA LPMO EnCNCh, Bt LPMO EnCNCh, and Ac CNCh.

| | To | Td | Char residue |
|---|---|---|---|
| Sample | (° C,) | (° C.) | (wt %) |
| α-Chitin | 286.01 | 394 | 13.4 |
| LFACA EnCNCh | 170.73 | 374 | 20.06 |
| Bt EnCNCh | 176.22 | 374 | 18.25 |
| AcCNCh | 263.66 | 385 | 13.18 |

TABLE 4
Crystallinity index (CI %) and crystallite size of chitin, EnCNCh
produced with LFACA LPMO enzymatic hydrolysis, EnCNCh produces
with extant Bt LPMO enzymatic hydrolysis and AcCNC produced by
HCl hydrolysis. The CI % was calculated by Segal method and the
Scherrer's equation was used for the crystallite size.

| | CI | Crystallite size |
|---|---|---|
| Enzyme | (%) | (nm) |
| α-Chitin | 88 | 7.2 |
| LFACA EnCNCh | 89 | 5.8 |
| Bt EnCNCh | 90 | 6.3 |
| AcCNCh | 82 | 4.9 |

Overall, these results demonstrate that LPMO, either ancestral or modern, can produce EnCNCh with physical features similar to those of native chitin but chemically modified, adding carboxyl groups that might serve as functional groups. However, the ability of LFACA LPMO to produce EnCNCh more efficiently than the extant LPMO should be highlighted, to be used as a distinct high-tech biomaterial.

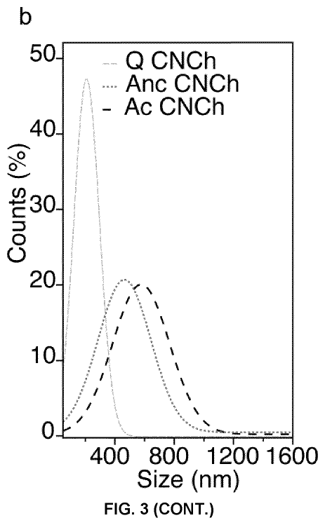

EnCNCh maintain native chitin features but also incorporates chemical modifications such as carboxyl groups. The superior efficiency of LFACA LPMO to produce EnCNCh brings the possibility of repurposing this new form of chitin for further applications. The inventors wondered whether the addition of chemical modification, the higher crystallinity, and the conservation of natural chitin structure but with nanometer size could be properties that would allow EnCNCh to perform as a biocompatible matrix material for bioprinting, which would bring nanochitin as a novel material for tissue engineering with properties akin to nanocellulose. In fact, chitosan obtained from chitin is already in use for such applications. However, chitosan is often mixed with other components such as collagen to generate a stable matrix for bioprinting. Therefore, the possibility of having a single-component matrix for bioprinting makes EnCNCh potentially better than chitosan. To probe the ability of EnCNCh obtained with LFACA LPMO for bioprinting, a suspension was concentrated to 10% EnCNCh and loaded into a syringe extruder in a 3D printer (FIG. 5a). The printed pieces perfectly maintain the shape of programmed form.

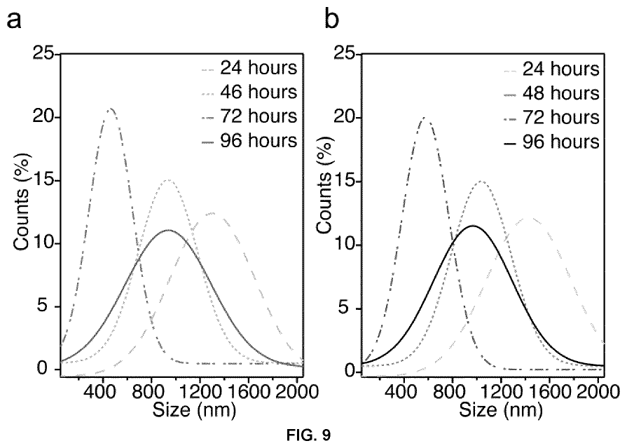

Conversely, CNCh obtained with chemical treatment was unable to form such stable scaffolds. HEK293T cells were seeded onto the EnCNCh scaffold and let proliferate for 3 days. Cells were dyed with DAPI and CellOrange to observe both, nucleus and cytoplasm of living cells that migrated and proliferated within the EnCNCh matrix (FIGS. 5b and 5c). Similarly, it was tested if EnCNCh would be able to assemble a stable 2D substrate for surface cell growth. EnCNCh films were prepared (FIG. 5d) that were stable submerged into water (FIG. 12) and cell culture media. HEK293T cells attached well to the EnCNCh substrate and were able to proliferate (FIGS. 5e and 5f). Interestingly, EnCNCh did not support Staphylococcus aureus attachment (FIGS. 5g and 5h), which is in line with the antibacterial activity reported for chitin.

The experiments above demonstrate that EnCNCh is a distinct material resulting from the physical and chemical conversion of chitin by LFACA LPMO that can serve as a matrix and substrate for cell proliferation. It was also wondered if EnCNCh could be mixed with other materials to create functional nanobiocomposites that potentiate the physical, chemical, and biological features of EnCNCh. Nanomaterials with controlled electrical properties are being investigated, and one key aspect that is being sought is their biocompatibility for biomedical applications. It was reasoned that a nanocomposite containing EnCNCh and graphene oxide would fulfill these requirements, i.e., able to form stable, printable, conductive and biocompatible films.

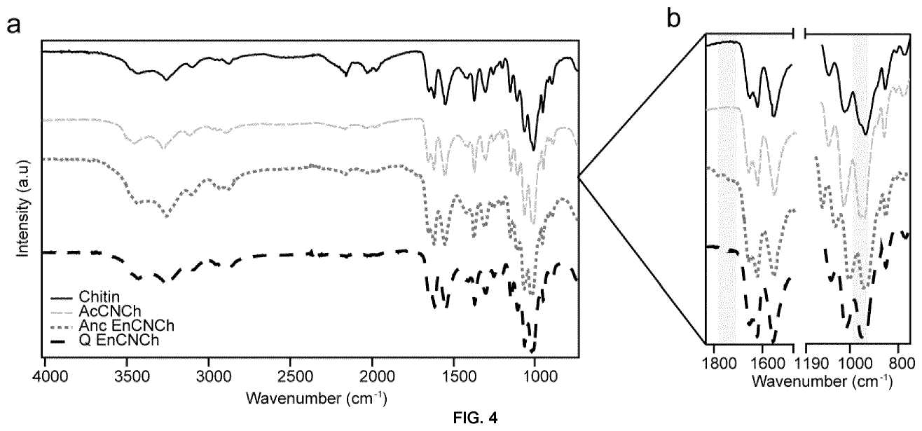

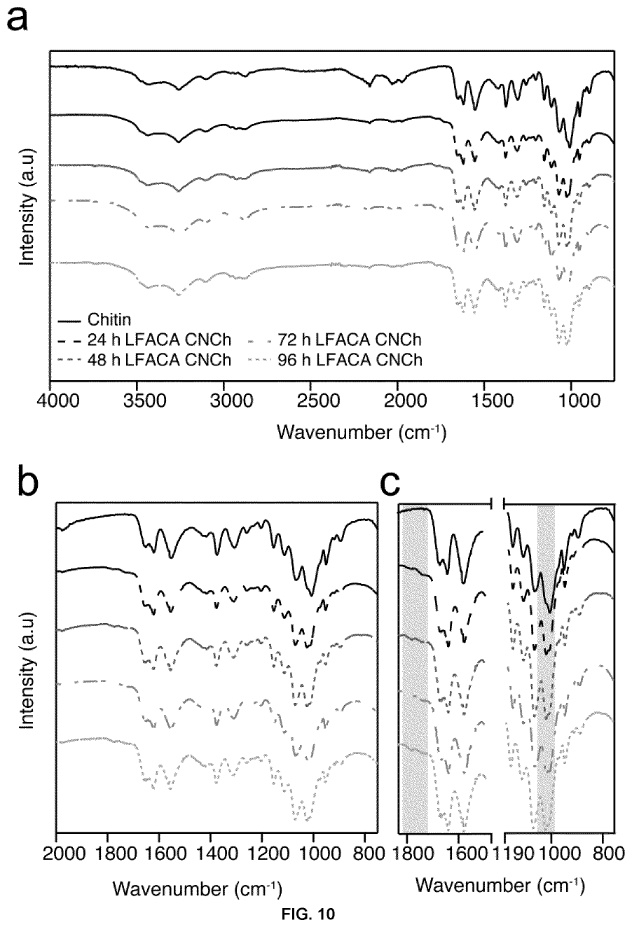

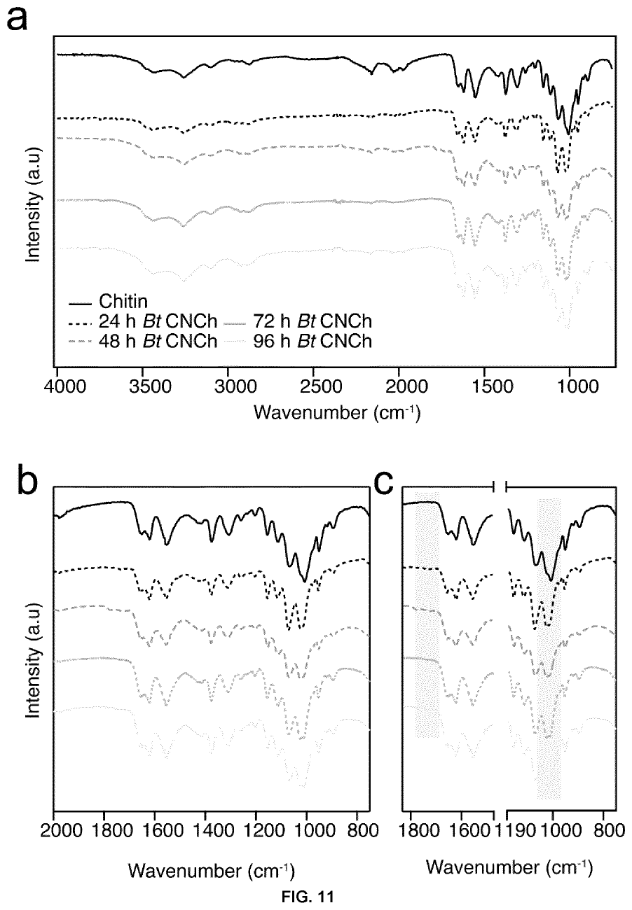

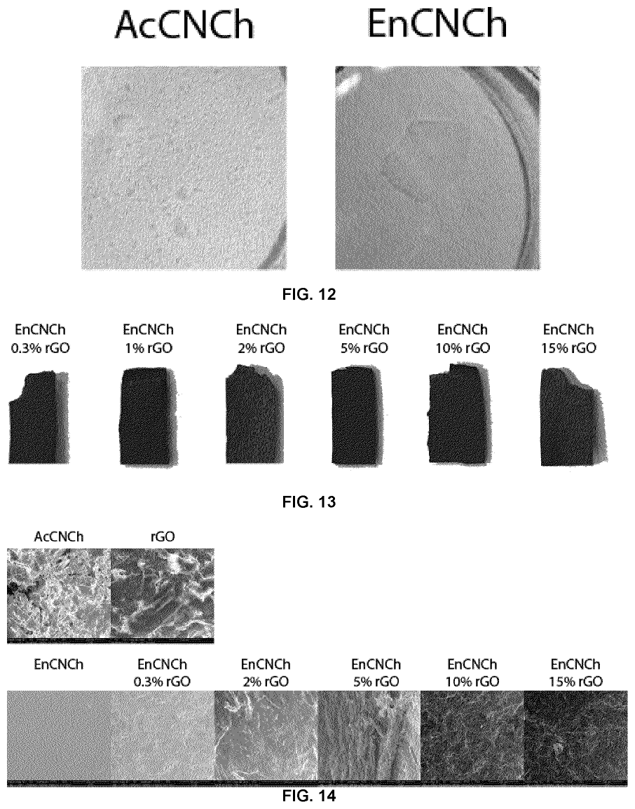

Hybrid EnCNCh films containing graphene oxide (GO) in different proportions from 0.3 to 15 wt % were prepared (FIG. 6a and FIG. 13). GO mixed well with EnCNCh and was chemically reduced after film assembly (rGO). Using SEM, the morphology and chemical composition of the reduced films was analyzed demonstrating the suitable dispersion of rGO in the EnCNCh matrix (FIG. 14). The chemical composition was also analyzed by FTIR (FIG. 15), and no chemical changes were observed in chitin as rGO % increased. The conductivity of the reduced films was analyzed observing an increasing conductivity with rGO content (Table 5). The conductivity reached a plateau of 11.1 S/m EnCNCh at around 10 wt % rGO content. Using these measurements, the electrical percolation threshold (ρc) was determined following the percolation theory. The conductivity was plotted against the volumetric rGO content (FIG. 6b) and calculated a ρc of 0.10% for EnCNCh. These value in this system corresponds to 0.25 wt % rGO, which is in the range of that determined for other graphene nanocomposites. On the contrary, AcCNCh failed to form stable films with rGO as they dispersed in water during the reduction step. The hybrid EnCNCh/rGO material could be perfectly used as conductive ink for printing (FIG. 6c). This is the first time that CNCh is used to create hybrid nanocomposites with graphene showing printing and conductive capabilities.

TABLE 5
Comparison of conductive properties of
EnCNCh films with reduced graphene.

| Sample | Conductivity (S/m) |
|---|---|
| EnCNCh | 0 |
| EnCNCh + 0.3% rGO | 8.8 × 10⁻⁵ ± 1.0 × 10⁻⁵ |
| EnCNCh + 1% rGO | 1.3 × 10⁻⁴ ± 3 × 10⁻⁵ |
| EnCNCh + 2% rGO | 2.8 ± 1.2 |
| EnCNCh + 5% rGO | 7.4 ± 2.1 |
| EnCNCh + 10% rGO | 11.1 ± 3.0 |
| EnCNCh + 15% rGO | 16.0 ± 2.4 |

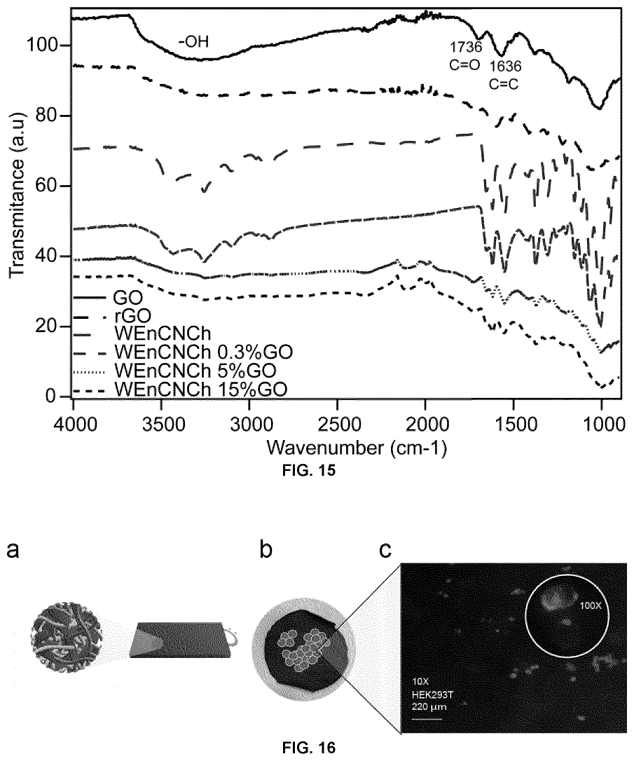

As mentioned above, these films were envisioned as the basis for obtaining conductive printable bioinks. A mixture of EnCNCh 10 wt % suspension was prepared and added 5 wt % GO, and used for printing. The printed ink was then freeze-dried, and finally, the scaffold was reduced. HEK293T cells were seeded into 3D printed scaffolds to test the viability of the hybrid material (FIG. 6d). After 3 days of culture, cells were dyed with DAPI and CellOrange dyes to observe both the nucleus and cytoplasm using fluorescence microscopy (FIG. 6e). The ability of EnCNCh/rGO films for 2D cell growth and proliferation was also tested by seeding films with the HEK293T cells (FIG. 16).

1-28. (canceled)

29. A polypeptide having lytic polysaccharide monooxygenase (LPMO) activity comprising the sequence of SEQ ID NO: 1 or a functionally equivalent variant thereof, wherein the functionally equivalent variant thereof has at least 65% sequence identity to the sequence of SEQ ID NO: 1 throughout the whole length of SEQ ID NO: 1.

30. The polypeptide according to claim 29, wherein the functionally equivalent variant has at least 70% sequence identity to the sequence of SEQ ID NO: 1 throughout the whole length of SEQ ID NO: 1.

31. A nucleic acid encoding a polypeptide according to claim 29, a vector comprising said nucleic acid or a host cell comprising said nucleic acid or said vector.

32. A method for degrading a polysaccharide comprising a β(1-4) glyosidic bond comprising contacting a sample containing said polysaccharide with a cooper-bound polypeptide having lytic polysaccharide monooxygenase (LPMO) activity under suitable conditions for the oxidative cleavage of said β(1-4) glycosidic bond, wherein the polypeptide having LPMO activity is the polypeptide according to claim 29.

33. The method according to claim 32, wherein the sample containing the polysaccharide is contacted with the polypeptide having LPMO activity in the presence of a reducing agent.

34. The method according to claim 32, wherein the polysaccharide is chitin, preferably α-chitin, or wherein the polysaccharide is cellulose.

35. The method according to claim 34, wherein if the polysaccharide is chitin, preferably α-chitin, the method further comprises contacting the sample containing chitin with a chitinase, or wherein if the polysaccharide is cellulose, the method further comprises contacting the sample containing cellulose with a cellulase.

36. A method for producing crystalline nanochitin comprising contacting a sample containing chitin with a copper-bound polypeptide having lytic polysaccharide monooxygenase (LPMO) activity under suitable conditions for the oxidative cleavage of chitin, wherein the polypeptide having LPMO activity is the polypeptide having LPMO activity according to claim 29.

37. The method according to claim 36, wherein the sample containing the polysaccharide is contacted with the polypeptide having LPMO activity in the presence of a reducing agent.

38. The method according to claim 36, wherein the chitin is α-chitin.

39. The method according to claim 36, wherein the suitable conditions for the oxidative cleavage of chitin by LPMO comprise contacting the sample containing chitin and the polypeptide having LPMO activity at a temperature between 30° C. and 90° C., or for a period of time between 24 and 96 hours.

40. The method according to claim 39, wherein the temperature is approximately 50° C. or wherein the period of time is approximately 72 hours.

41. The method according to claim 36, further comprising isolating the crystalline nanochitin.

42. The method according to claim 36, wherein the method does not comprise a step of contacting the sample containing chitin with a chitinase.

43. A composite material comprising:
(a) crystalline nanochitin, wherein said crystalline nanochitin is obtained by a method comprising contacting a sample comprising chitin with a copper-bound polypeptide having lytic polysaccharide monooxygenase activity (LPMO) under suitable conditions for the oxidative cleavage of chitin; and
(b) a carbon material selected from the group consisting of carbon nanotubes, carbon nanofibers, graphene, graphene oxide and a mixture thereof.

44. The composite material according to claim 43, wherein the polypeptide having LPMO activity comprises the sequence of SEQ ID NO: 1 or a functionally equivalent variant thereof, wherein the functionally equivalent variant thereof has at least 65% sequence identity to the sequence of SEQ ID NO: 1 throughout the whole length of SEQ ID NO: 1.

45. The composite material according to claim 43, wherein the composite material is an electrically conductive composite material.

46. A method for the preparation of the composite material according to claim 43, comprising the steps of:
(i) preparing a suspension comprising crystalline nanochitin and a carbon material selected from the group consisting of carbon nanotubes, carbon nanofibers, graphene, graphene oxide and a mixture thereof;
(ii) mixing the suspension; and
(iii) optionally reducing said carbon material in the mixture of the previous step.

47. A composite material obtainable by the method according to claim 46.
